(19) United States
(12) Patent Application Publication
(10) Pub. No.: US 2021/0123076 A1
(43) Pub. Date: Apr. 29, 2021

(54) ADENO-ASSOCIATED VIRUS (AAV)VECTORS FOR THE TREATMENT OF AGE-RELATED MACULAR DEGENERATION AND OTHER OCULAR DISEASES AND DISORDERS

(71) Applicant: Applied Genetic Technologies Corporation, Alachua, FL (US)

(72) Inventors: Mark Shearman, Alachua, FL (US); Adrian M Timmers, Burlington, MA (US); Judith Newmark, Milford, MA (US); Steven Pennock, Leominster, MA (US); Rakshaa Mureli, Gainesville, FL (US); Chunjuan Song, Chestbrook, PA (US); Lisa Keyes, Apex, NC (US)

(21) Appl. No.: 17/077,683

(22) Filed: Oct. 22, 2020

Related U.S. Application Data
(60) Provisional application No. 62/924,338, filed on Oct. 22, 2019.

(51) Int. Cl. C12N 15/86; A61P 27/02

(52) U.S. Cl. CPC C12N 15/86; C12N 2750/14111; A61P 27/02

(57) ABSTRACT

The present invention provides isolated promoters, transgene expression cassettes, vectors, kits, and methods for treatment of age-related macular generation and other genetic diseases that affect the cone cells of the retina.

### CROSS-REFERENCE TO RELATED APPLICATIONS

This application claims benefit under 35 U.S.C. § 119(e) of U.S. Provisional Application No. 62/924,338 filed Oct. 22, 2019, the contents of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 19, 2020, is named 119561-01702_SL.txt and is 48,600 bytes in size.

### FIELD OF THE INVENTION

The present invention relates to the field of gene therapy, including AAV vectors for expressing an isolated polynucleotides in a subject or cell. The disclosure also relates to nucleic acid constructs, promoters, vectors, and host cells including the polynucleotides as well as methods of delivering exogenous DNA sequences to a target cell, tissue, organ or organism, and methods for use in the treatment or prevention of age-related macular degeneration and other ocular diseases and disorders.

### BACKGROUND

Gene therapy aims to improve clinical outcomes for patients suffering from either genetic mutations or acquired diseases caused by an aberration in the gene expression profile. Gene therapy includes the treatment or prevention of medical conditions resulting from defective genes or abnormal regulation or expression, e.g. underexpression or overexpression, that can result in a disorder, disease, malignancy, etc. For example, a disease or disorder caused by a defective gene might be treated, prevented or ameliorated by delivery of a corrective genetic material to a patient, or might be treated, prevented or ameliorated by altering or silencing a defective gene, e.g., with a corrective genetic material to a patient resulting in the therapeutic expression of the genetic material within the patient.

The basis of gene therapy is to supply a transcription cassette with an active gene product (sometimes referred to as a transgene or a therapeutic nucleic acid), e.g., that can result in a positive gain-of-function effect, a negative loss-of-function effect, or another outcome. Such outcomes can be attributed to expression of a therapeutic protein such as an antibody, a functional enzyme, or a fusion protein. Gene therapy can also be used to treat a disease or malignancy caused by other factors. Human monogenic disorders can be treated by the delivery and expression of a normal gene to the target cells. Delivery and expression of a corrective gene in the patient's target cells can be carried out via numerous methods, including the use of engineered viruses and viral gene delivery vectors.

Adeno-associated viruses (AAV) belong to the Parvoviridae family and more specifically constitute the dependoparvovirus genus. Vectors derived from AAV (i.e., recombinant AAV (rAAV) or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the rep gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see ii), thus gaining persistence of the vector DNA and potentially, long-term expression of the therapeutic transgenes.

Age-related macular degeneration (AMD) is a leading cause of irreversible blindness in the elderly population in the developed world, affecting approximately 15% of individuals over the age of 60. An estimated 600 million individuals are in this age demographic. The prevalence of AMD increases with age; mild, or early forms occur in nearly 30%, and advanced forms in about 7%, of the population that is 75 years and older (Klein et al., Ophthalmol 1992; 99(6):933-943; Vingerling et al., Ophthalmol 1995 February; 102(2):205-210; Vingerling et al., Epidemiol Rev. 1995; 17(2):347-360). AMD is a late-onset, chronic and progressive degeneration of the retinal pigment epithelium (RPE) and photoreceptors at the macula. Clinically, AMD is characterized by a progressive loss of central vision attributable to degenerative changes that occur in the macula, a specialized region of the neural retina and underlying tissues. Early AMD is characterized by lipid and protein containing deposits (drusen), the hallmark ocular lesions associated with the onset of AMD, which occur between RPE and Bruch's membrane. Visual function is usually minimally disturbed at this stage but for changes in dark adaptation.

Several recent studies have reported an association between AMD and key proteins in the complement cascade. These studies have revealed the terminal pathway complement components (C5, C6, C7, C8 and C9) and activation-specific complement protein fragments of the terminal pathway (C3b, iC3b, C3dg and C5b-9) as well as various complement pathway regulators and inhibitors (including Factor H, Factor I, Factor D, CD55 and CD59) within drusen, along Bruch's membrane (an extracellular layer comprised of elastin and collagen that separates the RPE and the choroid) and within RPE cells overlying drusen (Johnson et al., Exp Eye Res. 2000; 70:441-449; Johnson et al., Exp. Eye Res. 2001; 73:887-896; Mullins et al. FASEB J. 2000; 14:835-846; Mullins et al., Eye 2001; 15:390-395). Mutations in complement-related genes including CFB, C2 and C3 have been associated with increased risk factor for AMD. However, polymorphisms in the CFH gene result in the greatest risk factor linked to AMD. For example, a tyrosine to histidine amino acid transition at position 402 in CFH, the key inhibitor of alternative pathway complement cascade C3 convertase, produces a nearly six-fold increase in the risk of AMD for individuals who harbor this Y402H polymorphism.

Factor H (FH) is a multifunctional protein that functions as a key regulator of the complement system (Zipfel, 2001. Semin Thromb Hemost. 27:191-9). The Factor H protein activities include: (1) binding to C-reactive protein (CRP), (2) binding to C3b, (3) binding to heparin, (4) binding to sialic acid; (5) binding to endothelial cell surfaces, (6) binding to cellular integrin receptors (7) binding to pathogens, including microbes, and (8) C3b co-factor activity. The Factor H gene, known as HF1, CFH and HF, is located on human chromosome 1, at position 1q32. The 1q32 particular locus contains a number of complement pathway-associated genes. One group of these genes, referred to as the regulators of complement activation (RCA) gene cluster, contains the genes that encode Factor H, five Factor H-related genes (FHR-1, FHR-2, FHR-3, FHR-4 and FHR-5 or CFHR1, CFHR2, CFHR3, CFHR4 and CFHR5, respectively), and the gene encoding the beta subunit of coagulation factor XIII. The Factor H and Factor H related genes are composed almost entirely of short consensus repeats (SCRs). A naturally occurring truncated form of CFH called Factor H-like protein 1 (FHL1) arises from alternative splicing of the CFH gene (Ripoche et al., Biochem J. 1988 Jan. 15; 249(2):593-602). FHL1 is identical to CFH for the first seven complement control protein (CCP) domains before terminating with a unique four amino acid C-terminus. FHL1 retains all the necessary domains for function and is also subject to the Y402H polymorphism. Previous studies have demonstrated FHL1 expression by RPE cells (Hageman et al., Proc Natl Acad Sci USA. 2005 May 17; 102(20):7227-32; Weinberger et al., Ophthalmic Res. 2014; 51(2):59-66). Factor H and FHL1, a natural occurring truncated variant form of CFH, are composed of SCRs 1-20 and 1-7, respectively.

The naturally occurring form of Factor H cDNA encodes a polypeptide 1231 amino acids in length having an apparent molecular weight of 155 kDa. cDNA and amino acid sequence data for human Factor H is found in the EMBL/GenBank Data Libraries under accession number Y00716.1. The naturally occurring truncated form of the human Factor H is found under GenBank accession number X07523.1.

Currently, there is no proven medical therapy for dry AMD, and no treatments are available for advanced dry AMD. Lampalizumab, a selective inhibitor against complement factor D, a rate-limiting enzyme (downstream of CFH activity) in the activation and amplification of the alternative complement pathway, dysfunction of which has been linked to the pathogenesis of AMD, failed to meet primary endpoints in stage III clinical trials.

AAV is a single-stranded, non-enveloped DNA virus that is a member of the parvovirus family. Different serotypes of AAV including AAV1, AAV2, AAV4, AAV5, AAV6, etc demonstrate different profiles of tissue distribution. The diverse tissue tropisms of these AAV capsids and capsid variants have enabled AAV based vectors to be used for widespread gene transfer applications both in vitro and in vivo for liver, skeletal muscle, brain, retina, heart and spinal cord (Wu, Z., et al., (2006) Molecular Therapy, 14: 316-327). AAV vectors can mediate long term gene expression in the retina and elicit minimal immune responses making these vectors an attractive choice for gene delivery to the eye. However, the optimal package capacity of AAV is 4.9-kb, and the size of the full length CFH cDNA containing all 20 complement-control protein modules (CCPs) is 3.69 kb. This leaves limited room for essential regulatory sequences such as promoter, poly adenylation (SV40 poly A) signal and the flanking AAV inverted terminal repeats (ITR).

The present disclosure addresses the need for effective treatment or prevention of ocular diseases and disorders, and in particular age-related macular degeneration, and further addresses the challenges of the size constraints of using the CFH gene in AAV therapeutics.

### SUMMARY OF THE INVENTION

CFH is a large gene which historically is too large to be used in AAV gene therapy when combined with all necessary elements. The present disclosure overcomes this challenge and describes engineered modifications of CFH cDNA that retain the biological functions of wild type CFH while fitting the CFH expression cassettes within the packaging capacity of rAAV (<4.9 kb). The technology described herein relates to methods and compositions for treatment or prevention of age-related macular degeneration and other ocular diseases and disorders by expression of CFH from a recombinant adeno-associated virus (rAAV) vector.

In a first aspect, the disclosure provides a nucleic acid encoding a truncated complement factor H (CFH) protein, wherein the truncated CFH protein comprises 5 or more complement control protein modules (CCPs) selected from the group consisting of CCP1, CCP2, CCP3, CCP4, CCP5, CCP6, CCP7, CCP8, CCP9, CCP10, CCP11, CCP12, CCP13, CCP14, CCP15, CCP16, CCP17, CCP18, CCP19 and CCP20.

According to some aspects, the disclosure provides a nucleic acid comprising a nucleotide sequence which is at least 85% identical to the nucleotide sequence of SEQ ID NO: 1. According to some embodiments, the nucleic acid encodes a CFH protein (tCFH1) comprising CCP1, CCP2, CCP3, CCP4, CCP5, CCP6, CCP7, CCP8, CCP9, CCP10, CCP11, CCP12, CCP13, CCP14, CCP15, CCP18, CCP19 and CCP20. According to some embodiments, the nucleic acid comprises SEQ ID NO: 1. According to some embodiments, the nucleic acid consists of SEQ ID NO: 1.

According to some aspects, the disclosure provides a nucleic acid comprising a nucleotide sequence which is at least 85% identical, at least 90% identical, at least 91% identical, at least 92% identical, at least 93% identical, at least 94% identical, at least 95% identical, at least 96% identical, at least 97% identical, at least 98% identical, at least 99% identical to the nucleotide sequence of SEQ ID NO: 2. According to some aspects, the disclosure provides a nucleic acid comprising a nucleotide sequence which is at least 85% identical, at least 90% identical, at least 91% identical, at least 92% identical, at least 93% identical, at least 94% identical, at least 95% identical, at least 96% identical, at least 97% identical, at least 98% identical, at least 99% identical to the nucleotide sequence of SEQ ID NO: 8. According to some embodiments, the nucleic acid encodes a CFH protein (tCFH1) comprising CCP1, CCP2, CCP3, CCP4, CCP5, CCP6, CCP7, CCP8, CCP9, CCP10, CCP11, CCP12, CCP13, CCP14, CCP15, CCP18, CCP19 and CCP20. According to some embodiments, the nucleic acid comprises SEQ ID NO: 2. According to some embodiments, the nucleic acid consists of SEQ ID NO: 2. According to some embodiments, the nucleic acid comprises SEQ ID NO: 8. According to some embodiments, the nucleic acid consists of SEQ ID NO: 8.

According to some aspects, the disclosure provides a nucleic acid comprising a nucleotide sequence which is at least 85% identical, at least 90% identical, at least 95% identical, at least 96% identical, at least 97% identical, at least 98% identical, at least 99% identical to the nucleotide sequence of SEQ ID NO: 3. According to some embodiments, the nucleic acid encodes a CFH protein (tCFH2) comprising CCP1, CCP2, CCP3, CCP4, CCP18, CCP19 and CCP20. According to some embodiments, the nucleic acid comprises SEQ ID NO: 3. According to some embodiments, the nucleic acid consists of SEQ ID NO: 3.

According to some aspects, the disclosure provides a nucleic acid comprising a nucleotide sequence which is at least 85% identical, at least 90% identical, at least 95% identical, at least 96% identical, at least 97% identical, at least 98% identical, at least 99% identical to the nucleotide sequence of SEQ ID NO: 4. According to some embodiments, the nucleic acid encodes a CFH protein (tCFH3) comprising CCP1, CCP2, CCP3, CCP4, CCP5, CCP6, CCP7, CCP8, CCP9, CCP16, CCP17, CCP18, CCP19 and CCP20. According to some embodiments, the nucleic acid comprises SEQ ID NO: 4. According to some embodiments, the nucleic acid consists of SEQ ID NO: 4.

According to some aspects, the disclosure provides a nucleic acid comprising a nucleotide sequence which is at least 85% identical, at least 90% identical, at least 95% identical, at least 96% identical, at least 97% identical, at least 98% identical, at least 99% identical to the nucleotide sequence of SEQ ID NO: 5. According to some embodiments, the nucleic acid encodes a CFH protein (tCFH4) comprising CCP1, CCP2, CCP3, CCP4, CCP5, CCP6, CCP7, CCP18, CCP19 and CCP20. According to some embodiments, the nucleic acid comprises SEQ ID NO: 5. According to some embodiments, the nucleic acid consists of SEQ ID NO: 5.

According to some aspects, the disclosure provides a nucleic acid comprising a nucleotide sequence which is at least 85% identical, at least 90% identical, at least 95% identical, at least 96% identical, at least 97% identical, at least 98% identical, at least 99% identical to the nucleotide sequence of SEQ ID NO: 6. According to some aspects, the disclosure features a nucleic acid consisting of the nucleotide sequence of SEQ ID NO: 6. According to some embodiments, the nucleic acid consists of SEQ ID NO: 6.

According to some aspects, the disclosure provides a transgene expression cassette comprising a promoter, the nucleic acid of any one of the aspects and embodiments herein, and minimal regulatory elements. According to some embodiments, the nucleic acid is a human nucleic acid. According to some embodiments, the disclosure provides a nucleic acid vector comprising the expression cassette of any of the aspects or embodiments herein. According to some embodiments, the vector is an adeno-associated viral (AAV) vector. According to some embodiments, the serotype of the capsid sequence and the serotype of the ITRs of said AAV vector are independently selected from the group consisting of AAV1, AAV2, AAV3, AAV4, AAV5, AAV6, AAV7, AAV8, AAV9, AAV10, AAV11, and AAV12. According to some embodiments, the serotype of the capsid sequence is AAV2. According to some embodiments, the capsid sequence is a mutant capsid sequence.

According to some aspects, the disclosure provides a mammalian cell comprising the vector of any one of the aspects or embodiments herein.

According to some aspects, the disclosure provides a method of making a recombinant adeno-associated viral (rAAV) vector comprising inserting into an adeno-associated viral vector a promoter and the nucleic acid of any one of the aspects or embodiments herein. According to some embodiments, the nucleic acid is a human nucleic acid. According to some embodiments, the serotype of the capsid sequence and the serotype of the ITRs of said AAV vector are independently selected from the group consisting of AAV1, AAV2, AAV3, AAV4, AAV5, AAV6, AAV7, AAV8, AAV9, AAV10, AAV11, and AAV12. According to some embodiments, the capsid sequence is a mutant capsid sequence.

According to some aspects, the disclosure provides a method of treating an ocular disease or disorder, comprising administering to a subject in need thereof the vector of any one of the aspects or embodiments herein, thereby treating the ocular disease or disorder in the subject. According to some embodiments, the ocular disease or disorder is associated with activation of the complement pathway. According to some embodiments, the ocular disease or disorder is retinal degeneration. According to some embodiments, the retinal degeneration is age related macular degeneration (AMD). According to some embodiments, the AMD is wet AMD. According to some embodiments, the AMD is dry AMD. According to some embodiments, the dry AMD is advanced dry AMD. According to some embodiments, the disclosure provides a method of preventing an ocular disease or disorder, comprising administering to a subject in need thereof the vector of any one of the aspects or embodiments herein, thereby preventing the ocular disease or disorder in the subject. According to some embodiments, the ocular disease or disorder is associated with activation of the complement pathway. According to some embodiments, the ocular disease or disorder is retinal degeneration. According to some embodiments, the retinal degeneration is age related macular degeneration (AMD). According to some embodiments, the AMD is wet AMD. According to some embodiments, the ocular disease or disorder is geographic atrophy (GA).

According to some embodiments, the vector is administered by an ocular route of delivery. According to some embodiments, the vector is administered retinally. According to some embodiments, the vector is administered subretinally. According to some embodiments, the vector is administered suprachoroidally. According to some embodiments, the vector is administered intravitreally.

According to some aspects, the disclosure provides a method for delivering a heterologous nucleic acid to the eye of an individual comprising administering the vector of any one of the aspects and embodiments herein to the eye of the individual, for example to the subretina of the individual.

According to some aspects, the disclosure provides a kit comprising the vector of any one of the aspects and embodiments herein, and instructions for use. According to some embodiments, the kit further comprises a device for ocular delivery of the vector.

### BRIEF DESCRIPTION OF THE DRAWINGS

FIG. 1A is a schematic that shows the 20 complement control protein modules (CCPs) of full length human CFH (3696 bp). CCP modules are shown as ovals. Some CCPs have identified binding sites for other proteins as indicated. The construct pTR-CBA-flCFH comprises the full length human CFH. The high-risk polymorphism Y402H for AMD is located in CCP 7 which is also contained in the natural occurring variant FHL-1.

FIG. 1B is a schematic that shows CFH constructs that were engineered to have various CCP deleted. The construct pTR-smCBA-tCFH1 comprises the full length human CFH with CCP 16-17 deleted. The construct pTR-smCBA-tCFH2 comprises the full length human CFH with CCP 5-17 deleted. The construct pTR-smCBA-tCFH3 comprises the full length human CFH with CCP 10-15 deleted. The construct pTR-smCBA-tCFH4 comprises the full length human CFH with CCP 8-17 deleted. The construct pTR-CBA-FHL-1 comprises the natural occurring variant FHL-1. The two constructs, tCFH2 and tCFH4, were engineered to delete CCPs known to be important for complement cascade activity.

FIG. 2 is a graph that shows the expression of CFH variants following plasmid transfection of human embryonic kidney 293 (HEK293) cells. HEK293 cells were transfected with plasmids containing engineered CFH variants (pTR-CFH variants as shown in FIG. 1A). Conditioned media and cellular lysates were harvested 48 hours post transfection and stored at −80° C. until assayed. CFH concentration (ng/ml) was determined in the lysates.

FIG. 3 shows the results of Western blot with anti-C3/C3b antibody to assay cleavage of human complement component C3b (C3b) by the CFH variants. HEK293 cells were transfected and cellular lysates were stored as described in FIG. 2. FIG. 3 shows that efficient cleavage was observed in the tCFH1 lane (lane 6, shown in box). Cleavage was absent or low by CFH variants smCBA-tCFH2 and smCBA-tCFH4.

Based on the results, the following CFH variants were selected for AAV production: 1) pTR-smCBA-flCFH; 2) pTR-smCBA-tCFH1; 3) pTR-CBA-tCFH3; 4) pTR-CBA-FHL-1.

FIG. 4 is a graph that shows expression of CFH variants following AAV infection of HEK293 cells. HEK293 cells were infected with a multiplicity of infection (MOI) of 1×10⁴. Media was collected 72 hours post infection, and CFH concentration (ng/ml) was determined in the media. As shown in the graph, there was robust expression of the engineered CFH constructs 72 hours following AAV-CFH infection of HEK293 cells.

FIG. 5 shows the results of Western blot with anti-C3/C3b antibody to assay the cleavage of C3b by the CFH variants as detected in the cell media 72 hours after AAV infection as described in FIG. 4. As shown in FIG. 5, cleavage of C3b was most efficient in the case of FHL-1, followed by tCFH1 and flCFH. It is noted that a background level C3b cleavage artefact was observed in lane 18 (FBS); this artefact was not present in lane 17 (DMEM/FBS).

FIG. 6 is a table that shows the expression of tCFH1 or FHL-1 in cfh−/− mice after subretinal (SubR) injection. Both CFH variants FHL-1 and tCFH1 are expressed following subretinal dosing of rAAV vectors in cfh−/− mice. As shown in the results in the table, dose response in FHL-1 expression was observed. Some animals were negative for expression of FHL-1 or tCFH1, which might have been due to unsuccessful injections. Expression level of tCFH1 or FHL-1 in RPE/Choroid was found to be higher than the level in neural retina.

FIG. 7A and FIG. 7B show the results of Western blot to determine Factor B (FB) complement fixation (detection of FB) in cfh−/− mice injected with tCFH1 variant. FIG. 7A shows factor B fixation in tCFH1 injected cfh−/− mice. FIG. 7B shows tCFH1 and FHL-1 expression. The results shown in FIG. 7A and FIG. 7B show that tCFH1 expression induced by rAAV-tCFH1 subretinal injection can fix factor B (FB) in RPE/Choroid. The CFH variant FHL-1 did not show FB fixation. These results support the biological functionality of tCFH1 expressed by rAAV and is the first time that AAV expressed CFH variants show complement fixation.

FIG. 8A and FIG. 8B show the results of electroretinogram (ERG) tests from cfh−/− mice injected with vehicle (FIG. 8A) and in cfh−/− mice injected with tCFH1 variant mid dose (FIG. 8B).

FIG. 9 shows the results of optical coherence tomography of in vivo, cross-sectional imagery of ocular tissues from left (injected) and right (uninjected) eyes for each of groups 1-6.

FIG. 10 shows the results of histological examination of ocular tissues on left (injected) and right (uninjected) eyes for each of groups 1-6.

FIG. 11 shows the results of Western blot to determine tCFH protein expression in cfh−/− mice injected with tCFH1 variant at low, mid and high doses.

FIG. 12 shows the results of Western blot to determine Factor B (FB) complement fixation (detection of FB) in cfh−/− mice injected with tCFH1 variant at various doses.

FIG. 13 shows the results from in vitro hemolytic experiments to evaluate the functionality of the rAAV-CFH variants.

### DETAILED DESCRIPTION

### I. Definitions

This disclosure is not limited to the particular methodology, protocols, cell lines, vectors, or reagents described herein because they may vary. Further, the terminology used herein is for the purpose of describing particular embodiments only and is not intended to limit the scope of the present disclosure.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present disclosure, the exemplary methods, devices, and materials are described herein.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The articles “a” and “an” are used herein to refer to one or to more than one (i.e. to at least one) of the grammatical object of the article. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

As used herein, the terms “administer,” “administering,” “administration,” and the like, are meant to refer to methods that are used to enable delivery of therapeutics or pharmaceutical compositions to the desired site of biological action. According to certain embodiments, these methods include subretinal injection, suprachoroidal injection or intravitreal injection to an eye.

As used herein, the term “carrier” is meant to include any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the terms “expression vector”, “vector” or “plasmid” can include any type of genetic construct, including AAV or rAAV vectors, containing a nucleic acid or polynucleotide coding for a gene product in which part or all of the nucleic acid encoding sequence is capable of being transcribed and is adapted for gene therapy. The transcript can be translated into a protein. In some instances, it may be partially translated or not translated. In certain embodiments, expression includes both transcription of a gene and translation of mRNA into a gene product. In other embodiments, expression only includes transcription of the nucleic acid encoding genes of interest. An expression vector can also comprise control elements operatively linked to the encoding region to facilitate expression of the protein in target cells. The combination of control elements and a gene or genes to which they are operably linked for expression can sometimes be referred to as an “expression cassette.”

As used herein, the term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence.

As used herein, the term “gene delivery” means a process by which foreign DNA is transferred to host cells for applications of gene therapy.

As used herein, the term “heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

As used herein, the term “increase,” “enhance,” “raise” (and like terms) generally refers to the act of increasing, either directly or indirectly, a concentration, level, function, activity, or behavior relative to the natural, expected, or average, or relative to a control condition.

As used herein, the term “inverted terminal repeat” or “ITR” sequence is meant to refer to relatively short sequences found at the termini of viral genomes which are in opposite orientation. An “AAV inverted terminal repeat (ITR)” sequence, a term well-understood in the art, is an approximately 145-nucleotide sequence that is present at both termini of the native single-stranded AAV genome. The outermost 145 nucleotides of the ITR can be present in either of two alternative orientations, leading to heterogeneity between different AAV genomes and between the two ends of a single AAV genome. The outermost 145 nucleotides also contain several shorter regions of self-complementarity (designated A, A′, B, B′, C, C′ and D regions), allowing intrastrand base-pairing to occur within this portion of the ITR.

A “wild-type ITR”, “WT-ITR” or “ITR” refers to the sequence of a naturally occurring ITR sequence in an AAV or other Dependovirus that retains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

As used herein, an “isolated” molecule (e.g., nucleic acid or protein) or cell means it has been identified and separated and/or recovered from a component of its natural environment.

As used herein, the term “minimal regulatory elements” is meant to refer to regulatory elements that are necessary for effective expression of a gene in a target cell and thus should be included in a transgene expression cassette. Such sequences could include, for example, promoter or enhancer sequences, a polylinker sequence facilitating the insertion of a DNA fragment within a plasmid vector, and sequences responsible for intron splicing and polyadenlyation of mRNA transcripts. In a recent example of a gene therapy treatment for achromatopsia, the expression cassette included the minimal regulatory elements of a polyadenylation site, splicing signal sequences, and AAV inverted terminal repeats. See, e.g., Komaromy et al. (Hum Mol Genet. 2010 Jul. 1; 19(13): 2581-2593).

As used herein, the term “minimize”, “reduce”, “decrease,” and/or “inhibit” (and like terms) generally refers to the act of reducing, either directly or indirectly, a concentration, level, function, activity, or behavior relative to the natural, expected, or average, or relative to a control condition.

As used herein, a “nucleic acid” or a “nucleic acid molecule” is meant to refer to a molecule composed of chains of monomeric nucleotides, such as, for example, DNA molecules (e.g., cDNA or genomic DNA). A nucleic acid may encode, for example, a promoter, the CFH gene or portion thereof, or regulatory elements. A nucleic acid molecule can be single-stranded or double-stranded. A “CFH nucleic acid” refers to a nucleic acid that comprises the CFH gene or a portion thereof, or a functional variant of the CFH gene or a portion thereof. A functional variant of a gene includes a variant of the gene with minor variations such as, for example, silent mutations, single nucleotide polymorphisms, missense mutations, and other mutations or deletions that do not significantly alter gene function.

The asymmetric ends of DNA and RNA strands are called the 5′ (five prime) and 3′ (three prime) ends, with the 5′ end having a terminal phosphate group and the 3′ end a terminal hydroxyl group. The five prime (5′) end has the fifth carbon in the sugar-ring of the deoxyribose or ribose at its terminus. Nucleic acids are synthesized in vivo in the 5′- to 3′-direction, because the polymerase used to assemble new strands attaches each new nucleotide to the 3′-hydroxyl (—OH) group via a phosphodiester bond.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure.

A DNA sequence that “encodes” a particular CFH protein (including fragments and portions thereof) is a nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the terms “operatively linked” or “operably linked” or “coupled” can refer to a juxtaposition of genetic elements, wherein the elements are in a relationship permitting them to operate in an expected manner. For instance, a promoter can be operatively linked to a coding region if the promoter helps initiate transcription of the coding sequence. There may be intervening residues between the promoter and coding region so long as this functional relationship is maintained.

As used herein, a “percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. An example of an alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

As used herein, the term “pharmaceutical composition” or “composition” is meant to refer to a composition or agent described herein (e.g. a recombinant adeno-associated (rAAV) expression vector), optionally mixed with at least one pharmaceutically acceptable chemical component, such as, though not limited to carriers, stabilizers, diluents, dispersing agents, suspending agents, thickening agents, excipients and the like.

As used herein, the terms “polypeptide” and “protein” are used interchangeably to refer to a polymer of amino acid residues and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature) to the native sequence, as long as the protein maintains the desired activity. These modifications may be deliberate, as through site-directed mutagenesis, or may be accidental, such as through mutations of hosts which produce the proteins or errors due to PCR amplification.

As used herein, a “promoter” is meant to refer to a region of DNA that facilitates the transcription of a particular gene. As part of the process of transcription, the enzyme that synthesizes RNA, known as RNA polymerase, attaches to the DNA near a gene. Promoters contain specific DNA sequences and response elements that provide an initial binding site for RNA polymerase and for transcription factors that recruit RNA polymerase. A “chicken beta-actin (CBA) promoter” refers to a polynucleotide sequence derived from a chicken beta-actin gene (e.g., Gallus gallus beta actin, represented by GenBank Entrez Gene ID 396526). A “smCBA” promoter refers to the small version of the hybrid CMV-chicken beta-actin promoter.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art.

As used herein, the term “recombinant” can refer to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, a “subject” or “patient” or “individual” to be treated by the method of the invention is meant to refer to either a human or non-human animal. A “nonhuman animal” includes any vertebrate or invertebrate organism. A human subject can be of any age, gender, race or ethnic group, e.g., Caucasian (white), Asian, African, black, African American, African European, Hispanic, Middle eastern, etc. In some embodiments, the subject can be a patient or other subject in a clinical setting. In some embodiments, the subject is already undergoing treatment. In some embodiments, the subject is a neonate, infant, child, adolescent, or adult.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

As used herein, the term “central retina”” refers to the outer macula and/or inner macula and/or the fovea. The term “central retina cell types” as used herein refers to cell types of the central retina, such as, for example, RPE and photoreceptor cells.

As used herein, the term “macula” refers to a region of the central retina in primates that contains a higher relative concentration of photoreceptor cells, specifically rods and cones, compared to the peripheral retina. The term “outer macula” as used herein may also be referred to as the “peripheral macula”. The term “inner macula” as used herein may also be referred to as the “central macula”.

As used herein, the term “fovea” is meant to refer to a small region in the central retina of primates of approximately equal to or less than 1.5 mm in diameter that contains a higher relative concentration of photoreceptor cells, specifically cones, when compared to the peripheral retina and the macula.

As used herein, the term “subretinal space” refers to the location in the retina between the photoreceptor cells and the retinal pigment epithelium cells. The subretinal space may be a potential space, such as prior to any subretinal injection of fluid. The subretinal space may also contain a fluid that is injected into the potential space. In this case, the fluid is “in contact with the subretinal space.” Cells that are “in contact with the subretinal space” include the cells that border the subretinal space, such as RPE and photoreceptor cells.

As used herein, the term “transgene” is meant to refer to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

A “transgene expression cassette” or “expression cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements. A transgene expression cassette comprises the gene sequences that a nucleic acid vector is to deliver to target cells. These sequences include the gene of interest (e.g., CFH nucleic acids or variants thereof), one or more promoters, and minimal regulatory elements.

As used herein, the term “treatment” or “treating” a disease or disorder (such as, for example, AMD) is meant to refer to alleviation of one or more signs or symptoms of the disease or disorder, diminishment of extent of disease or disorder, stabilized (e.g., not worsening) state of disease or disorder, preventing spread of disease or disorder, delay or slowing of disease or disorder progression, amelioration or palliation of the disease or disorder state, and remission (whether partial or total), whether detectable or undetectable. “Treatment” can also refer to prolonging survival as compared to expected survival if not receiving treatment.

As used herein, the term “vector” refers to a recombinant plasmid or virus that comprises a nucleic acid to be delivered into a host cell, either in vitro or in vivo.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, a “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

As used herein, a “recombinant AAV vector (rAAV vector)” refers to a polynucleotide vector comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”.

As used herein, a “rAAV virus” or “rAAV viral particle” refers to a viral particle composed of at least one AAV capsid protein and an encapsidated rAAV vector genome.

As used herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

Transcriptional regulators refer to transcriptional activators and repressors that either activate or repress transcription of a gene of interest, such as a truncated CFH, as described herein. Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “including” is used herein to mean, and is used interchangeably with, the phrase “including but not limited to.”

The term “such as” is used herein to mean, and is used interchangeably, with the phrase “such as but not limited to.”

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### II. Nucleic Acids

The characterization and development of nucleic acid molecules for potential therapeutic use are provided herein. The present disclosure provides promoters, expression cassettes, vectors, kits, and methods that can be used in the treatment of ocular diseases or disorders (e.g. age-related macular degeneration). Certain aspects of the disclosure relate to delivering a heterologous nucleic acid to an eye of a subject comprising administering a recombinant adeno-associated virus (rAAV) vector to the eye of the subject. According to some aspects, the disclosure provides methods of treating an ocular disease or disorder (e.g., age-related macular degeneration) comprising delivery of a composition comprising rAAV vectors described herein to the subject, wherein the rAAV vector comprises a heterologous nucleic acid (e.g. a nucleic acid encoding CFH) and further comprising two AAV terminal repeats. According to some embodiments, the heterologous nucleic acid is operably linked to a promoter.

Several genetic variants have been associated with AMD. The common coding variant Y402H in the complement factor H (CFH) gene was the first identified. The “CFH gene” is the gene that encodes the complement factor H (CFH) protein. CFH is a 155-kDa soluble glycoprotein regulator of the complement system. It is abundant in plasma and can associate with host cell membranes and other self-surfaces via recognition of polyanions such as glycosaminoglycans (GAGs) and sialic acid (Meri and Pangburn, Proc Natl Acad Sci USA. 1990 May; 87(10):3982-6). Through intervention at the level of the alternative-pathway C3 and C5 convertase enzymes it modulates both fluid-phase and surface-associated complement amplification. Factor H works in several ways (Pangburn et al. J Exp Med. 1977 Jul. 1; 146(1):257-70): it competes with factor B for binding to C3b, thus impeding formation of alternative-pathway C3 convertases (C3bBb); when bimolecular convertase complexes do succeed in assembling, CFH accelerates their subsequent dissociation (decay); CFH also accelerates decay of the alternative-pathway C5 convertase (C3b2Bb); and CFH is a co-factor for factor I-mediated proteolytic cleavage of C3b to iC3b. As a cofactor of the serine protease factor I, CFH also regulates proteolytic degradation of already-deposited C3b (Hocking et al., J. Biol. Chem. 283:9475-9487(2008); Xue et al., Nat. Struct. Mol. Biol. 24:643-651(2017)).

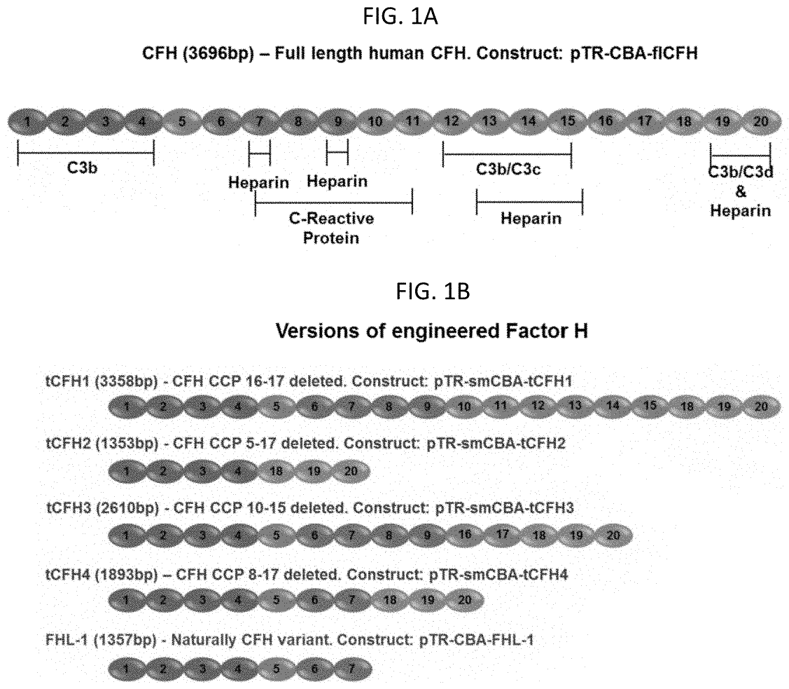

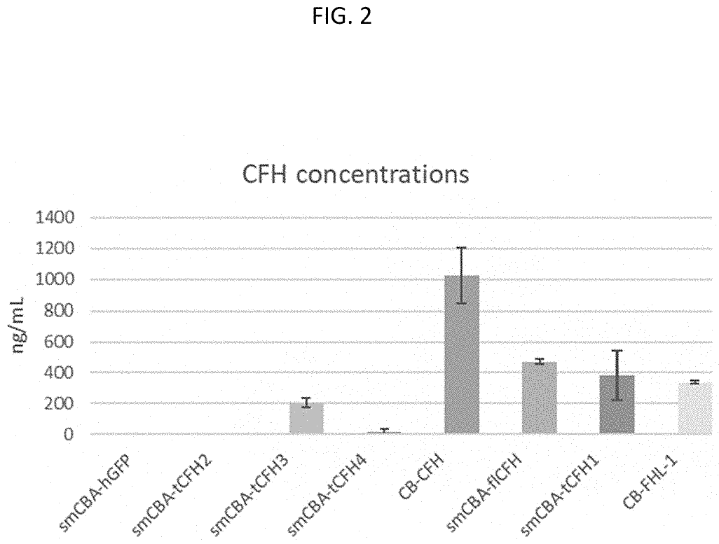

The 1213 amino acid residues of mature CFH (155 kDa) (Ripoche et al., Biochem J. 1988 Jan. 15; 249(2):593-602) consist of 20 short consensus repeats (SCRs), each of ˜60 residues (Kristensen and Tack. Proc Natl Acad Sci USA. 1986 June; 83(11):3963-7). A multiple alignment of the 20 SCRs shows four invariant Cys residues and a near-invariant Trp residue between Cys(III) and Cys(IV) (Schmidt et al., Clin Exp Immunol. 2008 January; 151(1): 14-24). Within CFH, ‘linkers’ of between three and eight residues lie between Cys(IV) (last residue) of one SCR and Cys(I) (first residue) of the next SCR. Each of the 20 SCRs (plus one or two residues within the linkers at either end) is presumed to fold into a distinct three-dimensional (3D) structure termed the complement control protein module (CCP) [(Soares and Barlow. Structural biology of the complement system. Boca Raton: CRC Press, Taylor & Francis Group; 2005. pp. 19-62), stabilized by Cys(I)-Cys(III), Cys(II)-Cys(IV) disulphide linkages. As shown in FIG. 1A, full length human CFH comprises 20 CCPs (CCPs 1-20). Some CCPs have identified binding sites for other proteins as indicated in FIG. 1A. The CCPs and CCP binding proteins play a critical role in complement cascade regulation. The high-risk polymorphism Y402H for AMD is located in CCP 7 which is also contained in the natural occurring variant FHL-1.

A “CFH nucleic acid” refers to a nucleic acid that comprises the CFH gene or a portion thereof, or a functional variant of the CFH gene or a portion thereof. A functional variant of a gene includes a variant of the gene with minor variations such as, for example, silent mutations, single nucleotide polymorphisms, missense mutations, and other mutations or deletions that do not significantly alter gene function.

According to some embodiments, a nucleic acid of the present invention encodes a CFH protein comprising complement control protein modules (CCPs) 1-20. According to some embodiments, a nucleic acid of the present invention encodes a CFH protein consisting of complement control protein modules (CCPs) 1-20. A truncated CFH protein is a CFH protein missing at least one, or a portion of one, of the 20 CCPs.

According to some embodiments, the expressed CFH protein is functional for the treatment of treatment of ocular diseases or disorders (e.g. the treatment and/or prevention of age-related macular degeneration). In some embodiments, expressed CFH protein does not cause an immune system reaction.

According to some embodiments, the nucleic acid sequence of full length CFH (comprising complement control protein modules (CCPs) 1-20) is shown below as SEQ ID NO: 1.

SEQ ID NO: 1
atgagacttctagcaaagattatttgccttatgttatgggctatttgtgt
agcagaagattgcaatgaacttcctccaagaagaaatacagaaattctga
caggttcctggtctgaccaaacatatccagaaggcacccaggctatctat
aaatgccgccctggatatagatctcttggaaatattataatggtatgcag
gaagggagaatgggttgctcttaatccattaaggaaatgtcagaaaaggc
cctgtggacatcctggagatactccttttggtacttttacccttacagga
ggaaatgtgtttgaatatggtgtaaaagctgtgtatacatgtaatgaggg
gtatcaattgctaggtgagattaattaccgtgaatgtgacacagatggat
ggaccaatgatattcctatatgtgaagttgtgaagtgtttaccagtgaca
gcaccagagaatggaaaaattgtcagtagtgcaatggaaccagatcggga
ataccattttggacaagcagtacggtttgtatgtaactcaggctacaaga
ttgaaggagatgaagaaatgcattgttcagacgatggtttttggagtaaa
gagaaaccaaagtgtgtggaaatttcatgcaaatccccagatgttataaa
tggatctcctatatctcagaagattatttataaggagaatgaacgatttc
aatataaatgtaacatgggttatgaatacagtgaaagaggagatgctgta
tgcactgaatctggatggcgtccgttgccttcatgtgaagaaaaatcatg
tgataatccttatattccaaatggtgactactcacctttaaggattaaac
acagaactggagatgaaatcacgtaccagtgtagaaatggtttttatcct
gcaacccggggaaatacagccaaatgcacaagtactggctggatacctgc
tccgagatgtaccttgaaaccttgtgattatccagacattaaacatggag
gtctatatcatgagaatatgcgtagaccatactttccagtagctgtagga
aaatattactcctattactgtgatgaacattttgagactccgtcaggaag
ttactgggatcacattcattgcacacaagatggatggtcgccagcagtac
catgcctcagaaaatgttattttccttatttggaaaatggatataatcaa
aattatggaagaaagtttgtacagggtaaatctatagacgttgcctgcca
tcctggctacgctcttccaaaagcgcagaccacagttacatgtatggaga
atggctggtctcctactcccagatgcatccgtgtcaaaacatgttccaaa
tcaagtatagatattgagaatgggtttatttctgaatctcagtatacata
tgccttaaaagaaaaagcgaaatatcaatgcaaactaggatatgtaacag
cagatggtgaaacatcaggatcaattagatgtgggaaagatggatggtca
gctcaacccacgtgcattaaatcttgtgatatcccagtatttatgaatgc
cagaactaaaaatgacttcacatggtttaagctgaatgacacattggact
atgaatgccatgatggttatgaaagcaatactggaagcaccactggttcc
atagtgtgtggttacaatggttggtctgatttacccatatgttatgaaag
agaatgcgaacttcctaaaatagatgtacacttagttcctgatcgcaaga
aagaccagtataaagttggagaggtgttgaaattctcctgcaaaccagga
tttacaatagttggacctaattccgttcagtgctaccactttggattgtc
tcctgacctcccaatatgtaaagagcaagtacaatcatgtggtccacctc
ctgaactcctcaatgggaatgttaaggaaaaaacgaaagaagaatatgga
cacagtgaagtggtggaatattattgcaatcctagatttctaatgaaggg
acctaataaaattcaatgtgttgatggagagtggacaactttaccagtgt
gtattgtggaggagagtacctgtggagatatacctgaacttgaacatggc
tgggcccagctttcttcccctccttattactatggagattcagtggaatt
caattgctcagaatcatttacaatgattggacacagatcaattacgtgta
ttcatggagtatggacccaacttccccagtgtgtggcaatagataaactt
aagaagtgcaaatcatcaaatttaattatacttgaggaacatttaaaaaa
caagaaggaattcgatcataattctaacataaggtacagatgtagaggaa
aagaaggatggatacacacagtctgcataaatggaagatgggatccagaa
gtgaactgctcaatggcacaaatacaattatgcccacctccacctcagat
tcccaattctcacaatatgacaaccacactgaattatcgggatggagaaa
aagtatctgttctttgccaagaaaattatctaattcaggaaggagaagaa
attacatgcaaagatggaagatggcagtcaataccactctgtgttgaaaa
aattccatgttcacaaccacctcagatagaacacggaaccattaattcat
ccaggtcttcacaagaaagttatgcacatgggactaaattgagttatact
tgtgagggtggtttcaggatatctgaagaaaatgaaacaacatgctacat
gggaaaatggagttctccacctcagtgtgaaggccttccttgtaaatctc
cacctgagatttctcatggtgttgtagctcacatgtcagacagttatcag
tatggagaagaagttacgtacaaatgttttgaaggttttggaattgatgg
gcctgcaattgcaaaatgcttaggagaaaaatggtctcaccctccatcat
gcataaaaacagattgtctcagtttacctagctttgaaaatgccataccc
atgggagagaagaaggatgtgtataaggcgggtgagcaagtgacttacac
ttgtgcaacatattacaaaatggatggagccagtaatgtaacatgcatta
atagcagatggacaggaaggccaacatgcagagacacctcctgtgtgaat
ccgcccacagtacaaaatgcttatatagtgtcgagacagatgagtaaata
tccatctggtgagagagtacgttatcaatgtaggagcccttatgaaatgt
ttggggatgaagaagtgatgtgtttaaatggaaactggacggaaccacct
caatgcaaagattctacaggaaaatgtgggccccctccacctattgacaa
tggggacattacttcattcccgttgtcagtatatgctccagcttcatcag
ttgagtaccaatgccagaacttgtatcaacttgagggtaacaagcgaata
acatgtagaaatggacaatggtcagaaccaccaaaatgcttacatccgtg
tgtaatatcccgagaaattatggaaaattataacatagcattaaggtgga
cagccaaacagaagctttattcgagaacaggtgaatcagttgaatttgtg
tgtaaacggggatatcgtctttcatcacgttctcacacattgcgaacaac
atgttgggatgggaaactggagtatccaacttgtgcaaaaagatag

According to some embodiments, the CFH nucleic acid comprises the nucleic acid sequence of SEQ ID NO: 1. According to some embodiments, the CFH nucleic acid consists of the nucleic acid sequence of SEQ ID NO: 1. According to some embodiments, the nucleic acid is at least 85% identical to SEQ ID NO: 1. According to some embodiments, the nucleic acid is at least 90% identical to SEQ ID NO: 1 According to some embodiments, the nucleic acid is at least 95%, 96%, 97%, or 98% identical to SEQ ID NO: 1. According to some embodiments, the nucleic acid is at least 99% identical to SEQ ID NO: 1.

According to some embodiments, a nucleic acid of the present invention encodes a truncated CFH protein comprising 5 or more complement control protein modules (CCPs) selected from the group consisting of: CCP1, CCP2, CCP3, CCP4, CCP5, CCP6, CCP7, CCP8, CCP9, CCP10, CCP11, CCP12, CCP13, CCP14, CCP15, CCP16, CCP17, CCP18, CCP19 and CCP20. According to some embodiments, a nucleic acid of the present invention encodes a truncated CFH protein comprising 7 or more complement control protein modules (CCPs) selected from the group consisting of: CCP1, CCP2, CCP3, CCP4, CCP5, CCP6, CCP7, CCP8, CCP9, CCP10, CCP11, CCP12, CCP13, CCP14, CCP15, CCP16, CCP17, CCP18, CCP19 and CCP20. According to some embodiments, a nucleic acid of the present invention encodes a truncated CFH protein comprising 10 or more complement control protein modules (CCPs) selected from the group consisting of: CCP1, CCP2, CCP3, CCP4, CCP5, CCP6, CCP7, CCP8, CCP9, CCP10, CCP11, CCP12, CCP13, CCP14, CCP15, CCP16, CCP17, CCP18, CCP19 and CCP20. According to some embodiments, a nucleic acid of the present invention encodes a truncated CFH protein comprising 15 or more complement control protein modules (CCPs) selected from the group consisting of: CCP1, CCP2, CCP3, CCP4, CCP5, CCP6, CCP7, CCP8, CCP9, CCP10, CCP11, CCP12, CCP13, CCP14, CCP15, CCP16, CCP17, CCP18, CCP19 and CCP20.

According to some embodiments, a nucleic acid of the present invention encodes a truncated CFH protein (tCFH1) comprising CCP1, CCP2, CCP3, CCP4, CCP5, CCP6, CCP7, CCP8, CCP9, CCP10, CCP11, CCP12, CCP13, CCP14, CCP15, CCP18, CCP19 and CCP20. According to some embodiments, a nucleic acid of the present invention encodes a truncated CFH protein consisting of CCP1, CCP2, CCP3, CCP4, CCP5, CCP6, CCP7, CCP8, CCP9, CCP10, CCP11, CCP12, CCP13, CCP14, CCP15, CCP18, CCP19 and CCP20. According to some embodiments, the nucleic acid sequence of a truncated CFH (tCFH1) is shown below as SEQ ID NO: 2.

SEQ ID NO: 2
atgagacttctagcaaagattatttgccttatgttatgggctatttgtgt
agcagaagattgcaatgaacttcctccaagaagaaatacagaaattctga
caggttcctggtctgaccaaacatatccagaaggcacccaggctatctat
aaatgccgccctggatatagatctcttggaaatattataatggtatgcag
gaagggagaatgggttgctcttaatccattaaggaaatgtcagaaaaggc
cctgtggacatcctggagatactccttttggtacttttacccttacagga
ggaaatgtgtttgaatatggtgtaaaagctgtgtatacatgtaatgaggg
gtatcaattgctaggtgagattaattaccgtgaatgtgacacagatggat
ggaccaatgatattcctatatgtgaagttgtgaagtgtttaccagtgaca
gcaccagagaatggaaaaattgtcagtagtgcaatggaaccagatcggga
ataccattttggacaagcagtacggtttgtatgtaactcaggctacaaga
ttgaaggagatgaagaaatgcattgttcagacgatggtttttggagtaaa
gagaaaccaaagtgtgtggaaatttcatgcaaatccccagatgttataaa
tggatctcctatatctcagaagattatttataaggagaatgaacgatttc
aatataaatgtaacatgggttatgaatacagtgaaagaggagatgctgta
tgcactgaatctggatggcgtccgttgccttcatgtgaagaaaaatcatg
tgataatccttatattccaaatggtgactactcacctttaaggattaaac
acagaactggagatgaaatcacgtaccagtgtagaaatggtttttatcct
gcaacccggggaaatacagccaaatgcacaagtactggctggatacctgc
tccgagatgtaccttgaaaccttgtgattatccagacattaaacatggag
gtctatatcatgagaatatgcgtagaccatactttccagtagctgtagga
aaatattactcctattactgtgatgaacattttgagactccgtcaggaag
ttactgggatcacattcattgcacacaagatggatggtcgccagcagtac
catgcctcagaaaatgttattttccttatttggaaaatggatataatcaa
aattatggaagaaagtttgtacagggtaaatctatagacgttgcctgcca
tcctggctacgctcttccaaaagcgcagaccacagttacatgtatggaga
atggctggtctcctactcccagatgcatccgtgtcaaaacatgttccaaa
tcaagtatagatattgagaatgggtttatttctgaatctcagtatacata
tgccttaaaagaaaaagcgaaatatcaatgcaaactaggatatgtaacag
cagatggtgaaacatcaggatcaattagatgtgggaaagatggatggtca
gctcaacccacgtgcattaaatcttgtgatatcccagtatttatgaatgc
cagaactaaaaatgacttcacatggtttaagctgaatgacacattggact
atgaatgccatgatggttatgaaagcaatactggaagcaccactggttcc
atagtgtgtggttacaatggttggtctgatttacccatatgttatgaaag
agaatgcgaacttcctaaaatagatgtacacttagttcctgatcgcaaga
aagaccagtataaagttggagaggtgttgaaattctcctgcaaaccagga
tttacaatagttggacctaattccgttcagtgctaccactttggattgtc
tcctgacctcccaatatgtaaagagcaagtacaatcatgtggtccacctc
ctgaactcctcaatgggaatgttaaggaaaaaacgaaagaagaatatgga
cacagtgaagtggtggaatattattgcaatcctagatttctaatgaaggg
acctaataaaattcaatgtgttgatggagagtggacaactttaccagtgt
gtattgtggaggagagtacctgtggagatatacctgaacttgaacatggc
tgggcccagctttcttcccctccttattactatggagattcagtggaatt
caattgctcagaatcatttacaatgattggacacagatcaattacgtgta
ttcatggagtatggacccaacttccccagtgtgtggcaatagataaactt
aagaagtgcaaatcatcaaatttaattatacttgaggaacatttaaaaaa
caagaaggaattcgatcataattctaacataaggtacagatgtagaggaa
aagaaggatggatacacacagtctgcataaatggaagatgggatccagaa
gtgaactgctcaatggcacaaatacaattatgcccacctccacctcagat
tcccaattctcacaatatgacaaccacactgaattatcgggatggagaaa
aagtatctgttctttgccaagaaaattatctaattcaggaaggagaagaa
attacatgcaaagatggaagatggcagtcaataccactctgtgttgaaaa
aattccatgttcacaaccacctcagatagaacacggaaccattaattcat
ccaggtcttcacaagaaagttatgcacatgggactaaattgagttatact
tgtgagggtggtttcaggatatctgaagaaaatgaaacaacatgctacat
gggaaaatggagttctccacctcagtgtgaaggccttggtacctcctgtg
tgaatccgcccacagtacaaaatgcttatatagtgtcgagacagatgagt
aaatatccatctggtgagagagtacgttatcaatgtaggagcccttatga
aatgtttggggatgaagaagtgatgtgtttaaatggaaactggacggaac
cacctcaatgcaaagattctacaggaaaatgtgggccccctccacctatt
gacaatggggacattacttcattcccgttgtcagtatatgctccagcttc
atcagttgagtaccaatgccagaacttgtatcaacttgagggtaacaagc
gaataacatgtagaaatggacaatggtcagaaccaccaaaatgcttacat
ccgtgtgtaatatcccgagaaattatggaaaattataacatagcattaag
gtggacagccaaacagaagctttattcgagaacaggtgaatcagttgaat
ttgtgtgtaaacggggatatcgtctttcatcacgttctcacacattgcga
acaacatgttgggatgggaaactggagtatccaacttgtgcaaaaagata
g

According to some embodiments, the nucleic acid sequence of a truncated CFH (tCFH1) is shown below as SEQ ID NO: 8.

ATGAGACTTCTAGCAAAGATTATTTGCCTTATGTTATGGGCTATTTGTGT
AGCAGAAGATTGCAATGAACTTCCTCCAAGAAGAAATACAGAAATTCTGA
CAGGTTCCTGGTCTGACCAAACATATCCAGAAGGCACCCAGGCTATCTAT
AAATGCCGCCCTGGATATAGATCTCTTGGAAATCGCCCTGGATATAGATC
TCTTGGAAATATCATAATGGTATGCAGGAAGGGAGAATGGGTTGCTCTTA
ATCCATTAAGGAAATGTCAGAAAAGGCCCTGTGGACATCCTGGAGATACT
CCTTTTGGTACTTTTACCCTTACAGGAGGAAATGTGTTTGAATATGGTGT
AAAAGCTGTGTATACATGTAATGAGGGGTATCAATTGCTAGGTGAGATTA
ATTACCGTGAATGTGACACAGATGGATGGACCAATGATATTCCTATATGT
GAAGTTGTGAAGTGTTTACCAGTGACAGCACCAGAGAATGGAAAAATTGT
CAGTAGTGCAATGGAACCAGATCGGGAATACCATTTTGGACAAGCAGTAC
GGTTTGTATGTAACTCAGGCTACAAGATTGAAGGAGATGAAGAAATGCAT
TGTTCAGACGATGGTTTTTGGAGTAAAGAGAAACCAAAGTGTGTGGAAAT
TTCATGCAAATCCCCAGATGTTATAAATGGATCTCCTATATCTCAGAAGA
TTATTTATAAGGAGAATGAACGATTTCAATATAAATGTAACATGGGTTAT
GAATACAGTGAAAGAGGAGATGCTGTATGCACTGAATCTGGATGGCGTCC
GTTGCCTTCATGTGAAGAAAAATCATGTGATAATCCTTATATTCCAAATG
GTGACTACTCACCTTTAAGGATTAAACACAGAACTGGAGATGAAATCACG
TACCAGTGTAGAAATGGTTTTTATCCTGCAACCCGGGGAAATACAGCAAA
ATGCACAAGTACTGGCTGGATACCTGCTCCGAGATGTACCTTGAAACCTT
GTGATTATCCAGACATTAAACATGGAGGTCTATATCATGAGAATATGCGT
AGACCATACTTTCCAGTAGCTGTAGGAAAATATTACTCCTATTACTGTGA
TGAACATTTTGAGACTCCGTCAGGAAGTTACTGGGATCACATTCATTGCA
CACAAGATGGATGGTCGCCAGCAGTACCATGCCTCAGAAAATGTTATTTT
CCTTATTTGGAAAATGGATATAATCAAAATTACGGAAGAAAGTTTGTACA
GGGTAAATCTATAGACGTTGCCTGCCATCCTGGCTACGCTCTTCCAAAAG
CGCAGACCACAGTTACATGTATGGAGAATGGCTGGTCTCCTACTCCCAGA
TGCATCCGTGTCAAAACATGTTCCAAATCAAGTATAGATATTGAGAATGG
GTTTATTTCTGAATCTCAGTATACATATGCCTTAAAAGAAAAAGCGAAAT
ATCAATGCAAACTAGGATATGTAACAGCAGATGGTGAAACATCAGGATCA
ATTACATGTGGGAAAGATGGATGGTCAGCTCAACCCACGTGCATTAAATC
TTGTGATATCCCAGTATTTATGAATGCCAGAACTAAAAATGACTTCACAT
GGTTTAAGCTGAATGACACATTGGACTATGAATGCCATGATGGTTATGAA
AGCAATACTGGAAGCACCACTGGTTCCATAGTGTGTGGTTACAATGGTTG
GTCTGATTTACCCATATGTTATGAAAGAGAATGCGAACTTCCTAAAATAG
ATGTACACTTAGTTCCTGATCGCAAGAAAGACCAGTATAAAGTTGGAGAG
GTGTTGAAATTCTCCTGCAAACCAGGATTTACAATAGTTGGACCTAATTC
CGTTCAGTGCTACCACTTTGGATTGTCTCCTGACCTCCCAATATGTAAAG
AGCAAGTACAATCATGTGGTCCACCTCCTGAACTCCTCAATGGGAATGTT
AAGGAAAAAACGAAAGAAGAATATGGACACAGTGAAGTGGTGGAATATTA
TTGCAATCCTAGATTTCTAATGAAGGGACCTAATAAAATTCAATGTGTTG
ATGGAGAGTGGACAACTTTACCAGTGTGTATTGTGGAGGAGAGTACCTGT
GGAGATATACCTGAACTTGAACATGGCTGGGCCCAGCTTTCTTCCCCTCC
TTATTACTATGGAGATTCAGTGGAATTCAATTGCTCAGAATCATTTACAA
TGATTGGACACAGATCAATTACGTGTATTCATGGAGTATGGACCCAACTT
CCCCAGTGTGTGGCAATAGATAAACTTAAGAAGTGCAAATCATCAAATTT
AATTATACTTGAGGAACATTTAAAAAACAAGAAGGAATTCGATCATAATT
CTAACATAAGGTACAGATGTAGAGGAAAAGAAGGATGGATACACACAGTC
TGCATAAATGGAAGATGGGATCCAGAAGTGAACTGCTCAATGGCACAAAT
ACAATTATGCCCACCTCCACCTCAGATTCCCAATTCTCACAATATGACAA
CCACACTGAATTATCGGGATGGAGAAAAAGTATCTGTTCTTTGCCAAGAA
AATTATCTAATTCAGGAAGGAGAAGAAATTACATGCAAAGATGGAAGATG
GCAGTCAATACCACTCTGTGTTGAAAAAATTCCATGTTCACAACCACCTC
AGATAGAACACGGAACCATTAATTCATCCAGGTCTTCACAAGAAAGTTAT
GCACATGGGACTAAATTGAGTTATACTTGTGAGGGTGGTTTCAGGATATC
TGAAGAAAATGAAACAACATGCTACATGGGAAAATGGAGTTCTCCACCTC
AGTGTGAAGGCACCTCCTGTGTGAATCCGCCCACAGTACAAAATGCTTAT
ATAGTGTCGAGACAGATGAGTAAATATCCATCTGGTGAGAGAGTACGTTA
TCAATGTAGGAGCCCTTATGAAATGTTTGGGGATGAAGAAGTGATGTGTT
TAAATGGAAACTGGACGGAACCACCTCAATGCAAAGATTCTACAGGAAAA
TGTGGGCCCCCTCCACCTATTGACAATGGGGACATTACTTCATTCCCGTT
GTCAGTATATGCTCCAGCTTCATCAGTTGAGTACCAATGCCAGAACTTGT
ATCAACTTGAGGGTAACAAGCGAATAACATGTAGAAATGGACAATGGTCA
GAACCACCAAAATGCTTACATCCGTGTGTAATATCCCGAGAAATTATGGA
AAATTATAACATAGCATTAAGGTGGACAGCCAAACAGAAGCTTTATTCGA
GAACAGGTGAATCAGTTGAATTTGTGTGTAAACGGGGATATCGTCTTTCA
TCACGTTCTCACACATTGCGAACAACATGTTGGGATGGGAAACTGGAGTA
TCCAACTTGTGCAAAAAGATAG

According to some embodiments, the nucleic acid comprises SEQ ID NO: 2. According to some embodiments, the nucleic acid comprises SEQ ID NO: 8. According to some embodiments, the nucleic acid consists of SEQ ID NO: 2. According to some embodiments, the nucleic acid consists of SEQ ID NO: 8. According to some embodiments, the nucleic acid is at least 85% identical to SEQ ID NO: 2. According to some embodiments, the nucleic acid is at least 85% identical to SEQ ID NO: 8. According to some embodiments, the nucleic acid is at least 90% identical to SEQ ID NO: 2. According to some embodiments, the nucleic acid is at least 90% identical to SEQ ID NO: 8. According to some embodiments, the nucleic acid is at least 95% identical to SEQ ID NO: 2. According to some embodiments, the nucleic acid is at least 95% identical to SEQ ID NO: 8. According to some embodiments, the nucleic acid is at least 96% identical to SEQ ID NO: 2. According to some embodiments, the nucleic acid is at least 96% identical to SEQ ID NO: 8. According to some embodiments, the nucleic acid is at least 97% identical to SEQ ID NO: 2. According to some embodiments, the nucleic acid is at least 97% identical to SEQ ID NO: 8. According to some embodiments, the nucleic acid is at least 98% identical to SEQ ID NO: 2. According to some embodiments, the nucleic acid is at least 98% identical to SEQ ID NO: 8. According to some embodiments, the nucleic acid is at least 99% identical to SEQ ID NO: 2. According to some embodiments, the nucleic acid is at least 99% identical to SEQ ID NO: 8.

According to some embodiments, a truncated CFH protein (tCFH1) comprises the amino acid sequence SEQ ID NO: 9, shown below.

SEQ ID NO: 9
MRLLAKIICLMLWAICVAEDCNELPPRRNTEILTGSWSDQTYPEGTQAIY
KCRPGYRSLGNIIMVCRKGEWVALNPLRKCQKRPCGHPGDTPFGTFTLTG
GNVFEYGVKAVYTCNEGYQLLGEINYRECDTDGWTNDIPICEVVKCLPVT
APENGKIVSSAMEPDREYHFGQAVRFVCNSGYKIEGDEEMHCSDDGFWSK
EKPKCVEISCKSPDVINGSPISQKIIYKENERFQYKCNMGYEYSERGDAV
CTESGWRPLPSCEEKSCDNPYIPNGDYSPLRIKHRTGDEITYQCRNGFYP
ATRGNTAKCTSTGWIPAPRCTLKPCDYPDIKHGGLYHENMRRPYFPVAVG
KYYSYYCDEHFETPSGSYWDHIHCTQDGWSPAVPCLRKCYFPYLENGYNQ
NYGRKFVQGKSIDVACHPGYALPKAQTTVTCMENGWSPTPRCIRVKTCSK
SSIDIENGFISESQYTYALKEKAKYQCKLGYVTADGETSGSIRCGKDGWS
AQPTCIKSCDIPVFMNARTKNDFTWFKLNDTLDYECHDGYESNTGSTTGS
IVCGYNGWSDLPICYERECELPKIDVHLVPDRKKDQYKVGEVLKFSCKPG
FTIVGPNSVQCYHFGLSPDLPICKEQVQSCGPPPELLNGNVKEKTKEEYG
HSEVVEYYCNPRFLMKGPNKIQCVDGEWTTLPVCIVEESTCGDIPELEHG
WAQLSSPPYYYGDSVEFNCSESFTMIGHRSITCIHGVWTQLPQCVAIDKL
KKCKSSNLIILEEHLKNKKEFDHNSNIRYRCRGKEGWIHTVCINGRWDPE
VNCSMAQIQLCPPPPQIPNSHNMTTTLNYRDGEKVSVLCQENYLIQEGEE
ITCKDGRWQSIPLCVEKIPCSQPPQIEHGTINSSRSSQESYAHGTKLSYT
CEGGFRISEENETTCYMGKWSSPPQCEGLGTSCVNPPTVQNAYIVSRQMS
KYPSGERVRYQCRSPYEMFGDEEVMCLNGNWTEPPQCKDSTGKCGPPPPI
DNGDITSFPLSVYAPASSVEYQCQNLYQLEGNKRITCRNGQWSEPPKCLH
PCVISREIMENYNIALRWTAKQKLYSRTGESVEFVCKRGYRLSSRSHTLR
TTCWDGKLEYPTCAKR

According to some embodiments, a truncated CFH protein (tCFH1) comprises the amino acid sequence SEQ ID NO: 10, shown below.

SEQ ID NO: 10
MRLLAKIICLMLWAICVAEDCNELPPRRNTEILTGSWSDQTYPEGTQAIY
KCRPGYRSLGNIIMVCRKGEWVALNPLRKCQKRPCGHPGDTPFGTFTLTG
GNVFEYGVKAVYTCNEGYQLLGEINYRECDTDGWTNDIPICEVVKCLPVT
APENGKIVSSAMEPDREYHFGQAVRFVCNSGYKIEGDEEMHCSDDGFWSK
EKPKCVEISCKSPDVINGSPISQKIIYKENERFQYKCNMGYEYSERGDAV
CTESGWRPLPSCEEKSCDNPYIPNGDYSPLRIKHRTGDEITYQCRNGFYP
ATRGNTAKCTSTGWIPAPRCTLKPCDYPDIKHGGLYHENMRRPYFPVAVG
KYYSYYCDEHFETPSGSYWDHIHCTQDGWSPAVPCLRKCYFPYLENGYNQ
NYGRKFVQGKSIDVACHPGYALPKAQTTVTCMENGWSPTPRCIRVKTCSK
SSIDIENGFISESQYTYALKEKAKYQCKLGYVTADGETSGSITCGKDGWS
AQPTCIKSCDIPVFMNARTKNDFTWFKLNDTLDYECHDGYESNTGSTTGS
IVCGYNGWSDLPICYERECELPKIDVHLVPDRKKDQYKVGEVLKFSCKPG
FTIVGPNSVQCYHFGLSPDLPICKEQVQSCGPPPELLNGNVKEKTKEEYG
HSEVVEYYCNPRFLMKGPNKIQCVDGEWTTLPVCIVEESTCGDIPELEHG
WAQLSSPPYYYGDSVEFNCSESFTMIGHRSITCIHGVWTQLPQCVAIDKL
KKCKSSNLIILEEHLKNKKEFDHNSNIRYRCRGKEGWIHTVCINGRWDPE
VNCSMAQIQLCPPPPQIPNSHNMTTTLNYRDGEKVSVLCQENYLIQEGEE
ITCKDGRWQSIPLCVEKIPCSQPPQIEHGTINSSRSSQESYAHGTKLSYT
CEGGFRISEENETTCYMGKWSSPPQCEGTSCVNPPTVQNAYIVSRQMSKY
PSGERVRYQCRSPYEMFGDEEVMCLNGNWTEPPQCKDSTGKCGPPPPIDN
GDITSFPLSVYAPASSVEYQCQNLYQLEGNKRITCRNGQWSEPPKCLHPC
VISREIMENYNIALRWTAKQKLYSRTGESVEFVCKRGYRLSSRSHTLRTT
CWDGKLEYPTCAKR

According to some embodiments, a truncated CFH protein (tCFH1) comprises an amino acid sequence at least 85% identical to SEQ ID NO: 9 or SEQ ID NO: 10. According to some embodiments, a truncated CFH protein (tCFH1) comprises an amino acid sequence at least 90% identical to SEQ ID NO: 9 or SEQ ID NO: 10. According to some embodiments, a truncated CFH protein (tCFH1) comprises an amino acid sequence at least 95% identical to SEQ ID NO: 9 or SEQ ID NO: 10. According to some embodiments, a truncated CFH protein (tCFH1) comprises an amino acid sequence at least 96% identical to SEQ ID NO: 9 or SEQ ID NO: 10. According to some embodiments, a truncated CFH protein (tCFH1) comprises an amino acid sequence at least 97% identical to SEQ ID NO: 9 or SEQ ID NO: 10. According to some embodiments, a truncated CFH protein (tCFH1) comprises an amino acid sequence at least 98% identical to SEQ ID NO: 9 or SEQ ID NO: 10. According to some embodiments, a truncated CFH protein (tCFH1) comprises an amino acid sequence at least 99% identical to SEQ ID NO: 9 or SEQ ID NO: 10. According to some embodiments, a truncated CFH protein (tCFH1) consists of SEQ ID NO: 9 or SEQ ID NO: 10.

According to some embodiments, a nucleic acid of the present invention encodes a truncated CFH protein (tCFH2) comprising CCP1, CCP2, CCP3, CCP4, CCP18, CCP19 and CCP20. According to some embodiments, a nucleic acid of the present invention encodes a truncated CFH protein consisting of CCP1, CCP2, CCP3, CCP4, CCP18, CCP19 and CCP20. According to some embodiments, the nucleic acid encoding the CFH protein is 1353 bp in length. According to some embodiments, the nucleic acid sequence of a truncated CFH (tCFH2) is shown below as SEQ ID NO: 3.

SEQ ID NO: 3
atgagacttctagcaaagattatttgccttatgttatgggctatttgtgta
gcagaagattgcaatgaacttcctccaagaagaaatacagaaattctgaca
ggttcctggtctgaccaaacatatccagaaggcacccaggctatctataaa
tgccgccctggatatagatctcttggaaatattataatggtatgcaggaag
ggagaatgggttgctcttaatccattaaggaaatgtcagaaaaggccctgt
ggacatcctggagatactccttttggtacttttacccttacaggaggaaat
gtgtttgaatatggtgtaaaagctgtgtatacatgtaatgaggggtatcaa
ttgctaggtgagattaattaccgtgaatgtgacacagatggatggaccaat
gatattcctatatgtgaagttgtgaagtgtttaccagtgacagcaccagag
aatggaaaaattgtcagtagtgcaatggaaccagatcgggaataccatttt
ggacaagcagtacggtttgtatgtaactcaggctacaagattgaaggagat
gaagaaatgcattgttcagacgatggtttttggagtaaagagaaaccaaag
tgtgtggaaatttcatgcaaatccccagatgttataaatggatctcctata
tctcagaagattatttataaggagaatgaacgatttcaatataaatgtaac
atgggttatgaatacagtgaaagaggagatgctgtatgcactgaatctgga
tggcgtccgttgccttcatgtgaagaaaaaggtacctcctgtgtgaatccg
cccacagtacaaaatgcttatatagtgtcgagacagatgagtaaatatcca
tctggtgagagagtacgttatcaatgtaggagcccttatgaaatgtttggg
gatgaagaagtgatgtgtttaaatggaaactggacggaaccacctcaatgc
aaagattctacaggaaaatgtgggccccctccacctattgacaatggggac
attacttcattcccgttgtcagtatatgctccagcttcatcagttgagtac
caatgccagaacttgtatcaacttgagggtaacaagcgaataacatgtaga
aatggacaatggtcagaaccaccaaaatgcttacatccgtgtgtaatatcc
cgagaaattatggaaaattataacatagcattaaggtggacagccaaacag
aagctttattcgagaacaggtgaatcagttgaatttgtgtgtaaacgggga
tatcgtctttcatcacgttctcacacattgcgaacaacatgttgggatggg
aaactggagtatccaacttgtgcaaaaagatag

According to some embodiments, the nucleic acid comprises SEQ ID NO: 3. According to some embodiments, the nucleic acid consists of SEQ ID NO: 3. According to some embodiments, the nucleic acid is at least 85% identical to SEQ ID NO: 3. According to some embodiments, the nucleic acid is at least 90% identical to SEQ ID NO: 3. According to some embodiments, the nucleic acid is at least 95%, 96%, 97%, or 98% identical to SEQ ID NO: 3. According to some embodiments, the nucleic acid is at least 99% identical to SEQ ID NO: 3.

According to some embodiments, a nucleic acid of the present invention encodes a truncated CFH protein (tCFH3) comprising CCP1, CCP2, CCP3, CCP4, CCP5, CCP6, CCP7, CCP8, CCP9, CCP16, CCP17, CCP18, CCP19 and CCP20. According to some embodiments, a nucleic acid of the present invention encodes a truncated CFH protein consisting of CCP1, CCP2, CCP3, CCP4, CCP5, CCP6, CCP7, CCP8, CCP9, CCP16, CCP17, CCP18, CCP19 and CCP20. According to some embodiments, the nucleic acid encoding the CFH protein is 2610 bp in length. According to some embodiments, the nucleic acid sequence of a truncated CFH (tCFH3) is shown below as SEQ ID NO: 4.

SEQ ID NO: 4
atgagacttctagcaaagattatttgccttatgttatgggctatttgtgta
gcagaagattgcaatgaacttcctccaagaagaaatacagaaattctgaca
ggttcctggtctgaccaaacatatccagaaggcacccaggctatctataaa
tgccgccctggatatagatctcttggaaatattataatggtatgcaggaag
ggagaatgggttgctcttaatccattaaggaaatgtcagaaaaggccctgt
ggacatcctggagatactccttttggtacttttacccttacaggaggaaat
gtgtttgaatatggtgtaaaagctgtgtatacatgtaatgaggggtatcaa
ttgctaggtgagattaattaccgtgaatgtgacacagatggatggaccaat
gatattcctatatgtgaagttgtgaagtgtttaccagtgacagcaccagag
aatggaaaaattgtcagtagtgcaatggaaccagatcgggaataccatttt
ggacaagcagtacggtttgtatgtaactcaggctacaagattgaaggagat
gaagaaatgcattgttcagacgatggtttttggagtaaagagaaaccaaag
tgtgtggaaatttcatgcaaatccccagatgttataaatggatctcctata
tctcagaagattatttataaggagaatgaacgatttcaatataaatgtaac
atgggttatgaatacagtgaaagaggagatgctgtatgcactgaatctgga
tggcgtccgttgccttcatgtgaagaaaaatcatgtgataatccttatatt
ccaaatggtgactactcacctttaaggattaaacacagaactggagatgaa
atcacgtaccagtgtagaaatggtttttatcctgcaacccggggaaataca
gccaaatgcacaagtactggctggatacctgctccgagatgtaccttgaaa
ccttgtgattatccagacattaaacatggaggtctatatcatgagaatatg
cgtagaccatactttccagtagctgtaggaaaatattactcctattactgt
gatgaacattttgagactccgtcaggaagttactgggatcacattcattgc
acacaagatggatggtcgccagcagtaccatgcctcagaaaatgttatttt
ccttatttggaaaatggatataatcaaaattatggaagaaagtttgtacag
ggtaaatctatagacgttgcctgccatcctggctacgctcttccaaaagcg
cagaccacagttacatgtatggagaatggctggtctcctactcccagatgc
atccgtgtcaaaacatgttccaaatcaagtatagatattgagaatgggttt
atttctgaatctcagtatacatatgccttaaaagaaaaagcgaaatatcaa
tgcaaactaggatatgtaacagcagatggtgaaacatcaggatcaattaga
tgtgggaaagatggatggtcagctcaacccacgtgcattaaatcttgtgat
atcccagtatttatgaatgccagaactaaaaatgacttcacatggtttaag
ctgaatgacacattggactatgaatgccatgatggttatgaaagcaatact
ggaagcaccactggttccatagtgtgtggttacaatggttggtctgattta
cccatatgttatgaaagaggtaccccttgtaaatctccacctgagatttct
catggtgttgtagctcacatgtcagacagttatcagtatggagaagaagtt
acgtacaaatgttttgaaggttttggaattgatgggcctgcaattgcaaaa
tgcttaggagaaaaatggtctcaccctccatcatgcataaaaacagattgt
ctcagtttacctagctttgaaaatgccatacccatgggagagaagaaggat
gtgtataaggcgggtgagcaagtgacttacacttgtgcaacatattacaaa
atggatggagccagtaatgtaacatgcattaatagcagatggacaggaagg
ccaacatgcagagacacctcctgtgtgaatccgcccacagtacaaaatgct
tatatagtgtcgagacagatgagtaaatatccatctggtgagagagtacgt
tatcaatgtaggagcccttatgaaatgtttggggatgaagaagtgatgtgt
ttaaatggaaactggacggaaccacctcaatgcaaagattctacaggaaaa
tgtgggccccctccacctattgacaatggggacattacttcattcccgttg
tcagtatatgctccagcttcatcagttgagtaccaatgccagaacttgtat
caacttgagggtaacaagcgaataacatgtagaaatggacaatggtcagaa
ccaccaaaatgcttacatccgtgtgtaatatcccgagaaattatggaaaat
tataacatagcattaaggtggacagccaaacagaagctttattcgagaaca
ggtgaatcagttgaatttgtgtgtaaacggggatatcgtctttcatcacgt
tctcacacattgcgaacaacatgttgggatgggaaactggagtatccaact
tgtgcaaaaagatag

According to some embodiments, the nucleic acid comprises SEQ ID NO: 4. According to some embodiments, the nucleic acid consists of SEQ ID NO: 4. According to some embodiments, the nucleic acid is at least 85% identical to SEQ ID NO: 4. According to some embodiments, the nucleic acid is at least 90% identical to SEQ ID NO: 4. According to some embodiments, the nucleic acid is at least 95%, 96%, 97%, or 98% identical to SEQ ID NO: 4. According to some embodiments, the nucleic acid is at least 99% identical to SEQ ID NO: 4.

According to some embodiments, a nucleic acid of the present invention encodes a truncated CFH protein (tCFH4) comprising CCP1, CCP2, CCP3, CCP4, CCP5, CCP6, CCP7, CCP18, CCP19 and CCP20. According to some embodiments, a nucleic acid of the present invention encodes a truncated CFH protein consisting of CCP1, CCP2, CCP3, CCP4, CCP5, CCP6, CCP7, CCP18, CCP19 and CCP20. According to some embodiments, the nucleic acid encoding the CFH protein is 1893 bp in length. According to some embodiments, the nucleic acid sequence of a truncated CFH (tCFH4) is shown below as SEQ ID NO: 5.

SEQ ID NO: 5
atgagacttctagcaaagattatttgccttatgttatgggctatttgtgta
gcagaagattgcaatgaacttcctccaagaagaaatacagaaattctgaca
ggttcctggtctgaccaaacatatccagaaggcacccaggctatctataaa
tgccgccctggatatagatctcttggaaatattataatggtatgcaggaag
ggagaatgggttgctcttaatccattaaggaaatgtcagaaaaggccctgt
ggacatcctggagatactccttttggtacttttacccttacaggaggaaat
gtgtttgaatatggtgtaaaagctgtgtatacatgtaatgaggggtatcaa
ttgctaggtgagattaattaccgtgaatgtgacacagatggatggaccaat
gatattcctatatgtgaagttgtgaagtgtttaccagtgacagcaccagag
aatggaaaaattgtcagtagtgcaatggaaccagatcgggaataccatttt
ggacaagcagtacggtttgtatgtaactcaggctacaagattgaaggagat
gaagaaatgcattgttcagacgatggtttttggagtaaagagaaaccaaag
tgtgtggaaatttcatgcaaatccccagatgttataaatggatctcctata
tctcagaagattatttataaggagaatgaacgatttcaatataaatgtaac
atgggttatgaatacagtgaaagaggagatgctgtatgcactgaatctgga
tggcgtccgttgccttcatgtgaagaaaaatcatgtgataatccttatatt
ccaaatggtgactactcacctttaaggattaaacacagaactggagatgaa
atcacgtaccagtgtagaaatggtttttatcctgcaacccggggaaataca
gccaaatgcacaagtactggctggatacctgctccgagatgtaccttgaaa
ccttgtgattatccagacattaaacatggaggtctatatcatgagaatatg
cgtagaccatactttccagtagctgtaggaaaatattactcctattactgt
gatgaacattttgagactccgtcaggaagttactgggatcacattcattgc
acacaagatggatggtcgccagcagtaccatgcctcagaaaatgttatttt
ccttatttggaaaatggatataatcaaaattatggaagaaagtttgtacag
ggtaaatctatagacgttgcctgccatcctggctacgctcttccaaaagcg
cagaccacagttacatgtatggagaatggctggtctcctactcccagatgc
atccgtgtcggtaccacaggaaaatgtgggccccctccacctattgacaat
ggggacattacttcattcccgttgtcagtatatgctccagcttcatcagtt
gagtaccaatgccagaacttgtatcaacttgagggtaacaagcgaataaca
tgtagaaatggacaatggtcagaaccaccaaaatgcttacatccgtgtgta
atatcccgagaaattatggaaaattataacatagcattaaggtggacagcc
aaacagaagctttattcgagaacaggtgaatcagttgaatttgtgtgtaaa
cggggatatcgtctttcatcacgttctcacacattgcgaacaacatgttgg
gatgggaaactggagtatccaacttgtgcaaaaagatag

According to some embodiments, the nucleic acid comprises SEQ ID NO: 5. According to some embodiments, the nucleic acid consists of SEQ ID NO: 5. According to some embodiments, the nucleic acid is at least 85% identical to SEQ ID NO: 5. According to some embodiments, the nucleic acid is at least 90% identical to SEQ ID NO: 5. According to some embodiments, the nucleic acid is at least 95%, 96%, 97%, or 98% identical to SEQ ID NO: 5. According to some embodiments, the nucleic acid is at least 99% identical to SEQ ID NO: 5.

According to some embodiments, a nucleic acid of the present invention encodes a truncated CFH protein (FHL-1) comprising CCP1, CCP2, CCP3, CCP4, CCP5, CCP6 and CCP7. According to some embodiments, a nucleic acid of the present invention encodes a truncated CFH protein consisting of CCP1, CCP2, CCP3, CCP4, CCP5, CCP6 and CCP7. According to some embodiments, the nucleic acid encoding the CFH protein is 1357 bp in length. According to some embodiments, the nucleic acid sequence of a truncated CFH protein (FHL-1) is shown below as SEQ ID NO: 6.

SEQ ID NO: 6
atgagacttctagcaaagattatttgccttatgttatgggctatttgtgta
gcagaagattgcaatgaacttcctccaagaagaaatacagaaattctgaca
ggttcctggtctgaccaaacatatccagaaggcacccaggctatctataaa
tgccgccctggatatagatctcttggaaatattataatggtatgcaggaag
ggagaatgggttgctcttaatccattaaggaaatgtcagaaaaggccctgt
ggacatcctggagatactccttttggtacttttacccttacaggaggaaat
gtgtttgaatatggtgtaaaagctgtgtatacatgtaatgaggggtatcaa
ttgctaggtgagattaattaccgtgaatgtgacacagatggatggaccaat
gatattcctatatgtgaagttgtgaagtgtttaccagtgacagcaccagag
aatggaaaaattgtcagtagtgcaatggaaccagatcgggaataccatttt
ggacaagcagtacggtttgtatgtaactcaggctacaagattgaaggagat
gaagaaatgcattgttcagacgatggtttttggagtaaagagaaaccaaag
tgtgtggaaatttcatgcaaatccccagatgttataaatggatctcctata
tctcagaagattatttataaggagaatgaacgatttcaatataaatgtaac
atgggttatgaatacagtgaaagaggagatgctgtatgcactgaatctgga
tggcgtccgttgccttcatgtgaagaaaaatcatgtgataatccttatatt
ccaaatggtgactactcacctttaaggattaaacacagaactggagatgaa
atcacgtaccagtgtagaaatggtttttatcctgcaacccggggaaataca
gccaaatgcacaagtactggctggatacctgctccgagatgtaccttgaaa
ccttgtgattatccagacattaaacatggaggtctatatcatgagaatatg
cgtagaccatactttccagtagctgtaggaaaatattactcctattactgt
gatgaacattttgagactccgtcaggaagttactgggatcacattcattgc
acacaagatggatggtcgccagcagtaccatgcctcagaaaatgttatttt
ccttatttggaaaatggatataatcaaaattatggaagaaagtttgtacag
ggtaaatctatagacgttgcctgccatcctggctacgctcttccaaaagcg
cagaccacagttacatgtatggagaatggctggtctcctactcccagatgc
atccgtgtcagctttaccctctga

According to some embodiments, the nucleic acid comprises SEQ ID NO: 6. According to some embodiments, the nucleic acid consists of SEQ ID NO: 6. According to some embodiments, the nucleic acid is at least 85% identical to SEQ ID NO: 6. According to some embodiments, the nucleic acid is at least 90% identical to SEQ ID NO: 6. According to some embodiments, the nucleic acid is at least 95%, 96%, 97%, or 98% identical to SEQ ID NO: 6. According to some embodiments, the nucleic acid is at least 99% identical to SEQ ID NO: 6.

According to some embodiments, a nucleic acid of the present invention encodes a CFH protein with deletion of CCPs known to be important for complement cascade activity. According to some embodiments, tCFH2 and tCFH4, were engineered to delete CCPs known to be important for complement cascade activity.

According to certain embodiments, the nucleic acid is a human nucleic acid (i.e., a nucleic acid that is derived from a human CFH gene). In other embodiments, the nucleic acid is a non-human nucleic acid (i.e., a nucleic acid that is derived from a non-human CFH gene).

Making Nucleic Acids

A nucleic acid molecule (including, for example, a CFH nucleic acid) of the present invention can be isolated using standard molecular biology techniques. Using all or a portion of a nucleic acid sequence of interest as a hybridization probe, nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

A nucleic acid molecule for use in the methods of the invention can also be isolated by the polymerase chain reaction (PCR) using synthetic oligonucleotide primers designed based upon the sequence of a nucleic acid molecule of interest. A nucleic acid molecule used in the methods of the invention can be amplified using cDNA, mRNA or, alternatively, genomic DNA as a template and appropriate oligonucleotide primers according to standard PCR amplification techniques.

Furthermore, oligonucleotides corresponding to nucleotide sequences of interest can also be chemically synthesized using standard techniques. Numerous methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein). Automated methods for designing synthetic oligonucleotides are available. See e.g., Hoover, D. M. & Lubowski, 2002. J. Nucleic Acids Research, 30(10): e43.

Many embodiments of the invention involve a CFH nucleic acid. Some aspects and embodiments of the invention involve other nucleic acids, such as isolated promoters or regulatory elements. A nucleic acid may be, for example, a cDNA or a chemically synthesized nucleic acid. A cDNA can be obtained, for example, by amplification using the polymerase chain reaction (PCR) or by screening an appropriate cDNA library. Alternatively, a nucleic acid may be chemically synthesized.

### III. Promoter, Expression Cassettes and Vectors

The promoters, CFH nucleic acids, regulatory elements, and expression cassettes, and vectors of the disclosure may be produced using methods known in the art. The methods described below are provided as non-limiting examples of such methods.

Promoters

Expression of CFH proteins as described herein from an AAV vector can be achieved both spatially and temporally using one or more of the promoters as described herein.

Expression cassettes of the AAV vector for expression of CFH protein can include a promoter, which can influence overall expression levels. Exemplary promoters include, but are not limited to, the cytomegalovirus (CMV) immediate early promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the chicken beta-actin promoter, the small version of the hybrid CMV-chicken beta-actin promoter (smCBA) (Pang et al., Invest Ophthalmol Vis Sci. 2008 October; 49(10):4278-83); the a cytomegalovirus enhancer linked to a chicken beta-actin (CBA) promoter; the cytomegalovirus enhancer/chicken beta-actin/Rabbit beta-globin promoter (CAG promoter; Niwa et al., Gene, 1991, 108(2):193-9) and the elongation factor 1-alpha promoter (EF1-alpha) promoter (Kim et al., Gene, 1990, 91(2):217-23 and Guo et al., Gene Ther., 1996, 3(9):802-10). In some embodiments, the promoter comprises the chicken beta-actin promoter. According to some embodiments, the promoter comprises the small version of the hybrid CMV-chicken beta-actin promoter (smCBA). The promoter can be a constitutive, inducible or repressible promoter. In some embodiments, the promoter is capable of expressing the heterologous nucleic acid in a cell of the eye. In some embodiments, the promoter is capable of expressing the heterologous nucleic acid in photoreceptor cells or RPE. In some embodiments, the promoter is capable of expressing the heterologous nucleic acid in a multitude of retinal cells.

Expression Cassettes

In another aspect, the present invention provides a transgene expression cassette that includes (a) a promoter; (b) a nucleic acid comprising a CFH nucleic acid as described herein; and (c) minimal regulatory elements. A promoter of the invention includes the promoters discussed supra. According to some embodiments, the promoter is CBA. According to some embodiments, the promoter is smCBA.

According to some embodiments, a nucleic acid of the present invention encodes a truncated CFH protein (tCFH1) comprising CCP1, CCP2, CCP3, CCP4, CCP5, CCP6, CCP7, CCP8, CCP9, CCP10, CCP11, CCP12, CCP13, CCP14, CCP15, CCP18, CCP19 and CCP20. According to some embodiments, a nucleic acid of the present invention encodes a truncated CFH protein consisting of CCP1, CCP2, CCP3, CCP4, CCP5, CCP6, CCP7, CCP8, CCP9, CCP10, CCP11, CCP12, CCP13, CCP14, CCP15, CCP18, CCP19 and CCP20. According to some embodiments, the nucleic acid encoding the CFH protein is 3358 bp in length. According to some embodiments, the nucleic acid comprises SEQ ID NO: 2. According to some embodiments, the nucleic acid consists of SEQ ID NO: 2. According to some embodiments, the nucleic acid is at least 85% identical to SEQ ID NO: 2. According to some embodiments, the nucleic acid is at least 90% identical to SEQ ID NO: 2. According to some embodiments, the nucleic acid is at least 95%, 96%, 97%, or 98% identical to SEQ ID NO: 2. According to some embodiments, the nucleic acid is at least 99% identical to SEQ ID NO: 2. According to some embodiments, the nucleic acid comprises SEQ ID NO: 8. According to some embodiments, the nucleic acid consists of SEQ ID NO: 8. According to some embodiments, the nucleic acid is at least 85% identical to SEQ ID NO: 8. According to some embodiments, the nucleic acid is at least 90% identical to SEQ ID NO: 8. According to some embodiments, the nucleic acid is at least 95%, 96%, 97%, or 98% identical to SEQ ID NO: 8. According to some embodiments, the nucleic acid is at least 99% identical to SEQ ID NO: 8.

According to some embodiments, a nucleic acid of the present invention encodes a truncated CFH protein (tCFH2) comprising CCP1, CCP2, CCP3, CCP4, CCP18, CCP19 and CCP20. According to some embodiments, a nucleic acid of the present invention encodes a truncated CFH protein consisting of CCP1, CCP2, CCP3, CCP4, CCP18, CCP19 and CCP20. According to some embodiments, the nucleic acid encoding the CFH protein is 1353 bp in length. According to some embodiments, the nucleic acid comprises SEQ ID NO: 3. According to some embodiments, the nucleic acid consists of SEQ ID NO: 3. According to some embodiments, the nucleic acid is at least 85% identical to SEQ ID NO: 3. According to some embodiments, the nucleic acid is at least 90% identical to SEQ ID NO: 3. According to some embodiments, the nucleic acid is at least 95%, 96%, 97%, or 98% identical to SEQ ID NO: 3. According to some embodiments, the nucleic acid is at least 99% identical to SEQ ID NO: 3.

According to some embodiments, a nucleic acid of the present invention encodes a truncated CFH protein (tCFH3) comprising CCP1, CCP2, CCP3, CCP4, CCP5, CCP6, CCP7, CCP8, CCP9, CCP16, CCP17, CCP18, CCP19 and CCP20. According to some embodiments, a nucleic acid of the present invention encodes a truncated CFH protein consisting of CCP1, CCP2, CCP3, CCP4, CCP5, CCP6, CCP7, CCP8, CCP9, CCP16, CCP17, CCP18, CCP19 and CCP20. According to some embodiments, the nucleic acid encoding the CFH protein is 2610 bp in length. According to some embodiments, the nucleic acid comprises SEQ ID NO: 4. According to some embodiments, the nucleic acid consists of SEQ ID NO: 4. According to some embodiments, the nucleic acid is at least 85% identical to SEQ ID NO: 4. According to some embodiments, the nucleic acid is at least 90% identical to SEQ ID NO: 4. According to some embodiments, the nucleic acid is at least 95%, 96%, 97%, or 98% identical to SEQ ID NO: 4. According to some embodiments, the nucleic acid is at least 99% identical to SEQ ID NO: 4.

According to some embodiments, a nucleic acid of the present invention encodes a truncated CFH protein (tCFH4) comprising CCP1, CCP2, CCP3, CCP4, CCP5, CCP6, CCP7, CCP18, CCP19 and CCP20. According to some embodiments, a nucleic acid of the present invention encodes a truncated CFH protein consisting of CCP1, CCP2, CCP3, CCP4, CCP5, CCP6, CCP7, CCP18, CCP19 and CCP20. According to some embodiments, the nucleic acid encoding the CFH protein is 1893 bp in length. According to some embodiments, the nucleic acid comprises SEQ ID NO: 5. According to some embodiments, the nucleic acid consists of SEQ ID NO: 5. According to some embodiments, the nucleic acid is at least 85% identical to SEQ ID NO: 5. According to some embodiments, the nucleic acid is at least 90% identical to SEQ ID NO: 5. According to some embodiments, the nucleic acid is at least 95%, 96%, 97%, or 98% identical to SEQ ID NO: 5. According to some embodiments, the nucleic acid is at least 99% identical to SEQ ID NO: 5.

According to some embodiments, a nucleic acid of the present invention encodes a truncated CFH protein (FHL-1) comprising CCP1, CCP2, CCP3, CCP4, CCP5, CCP6 and CCP7. According to some embodiments, a nucleic acid of the present invention encodes a truncated CFH protein consisting of CCP1, CCP2, CCP3, CCP4, CCP5, CCP6 and CCP7. According to some embodiments, the nucleic acid encoding the CFH protein is 1357 bp in length. According to some embodiments, the nucleic acid comprises SEQ ID NO: 6. According to some embodiments, the nucleic acid consists of SEQ ID NO: 6. According to some embodiments, the nucleic acid is at least 85% identical to SEQ ID NO: 6. According to some embodiments, the nucleic acid is at least 90% identical to SEQ ID NO: 6. According to some embodiments, the nucleic acid is at least 95%, 96%, 97%, or 98% identical to SEQ ID NO: 6. According to some embodiments, the nucleic acid is at least 99% identical to SEQ ID NO: 6.

According to some embodiments, the recombinant nucleic acid is flanked by at least two ITRs.

According to some embodiments, the construct comprises full length human CFH, chicken beta actin promoter and inverted terminal repeats (pTR-CBA-flCFH).

According to some embodiments, the construct comprises full length human CFH with CFH CCP 16-17 deleted, the small version of the hybrid CMV-chicken beta-actin promoter and inverted terminal repeats (pTR-smCBA-tCFH1).

According to some embodiments, the construct comprises full length human CFH with CFH CCP 5-17 deleted, the small version of the hybrid CMV-chicken beta-actin promoter and inverted terminal repeats (pTR-smCBA-tCFH2).

According to some embodiments, the construct comprises full length human CFH with CFH CCP 10-15 deleted, the small version of the hybrid CMV-chicken beta-actin promoter and inverted terminal repeats (pTR-smCBA-tCFH3).

According to some embodiments, the construct comprises full length human CFH with CFH CCP 8-17 deleted, the small version of the hybrid CMV-chicken beta-actin promoter and inverted terminal repeats (pTR-smCBA-tCFH4).

According to some embodiments, the construct comprises a naturally occurring CFH variant comprising CCPs 1-7, the chicken beta-actin promoter and inverted terminal repeats (pTR-CBA-FHL-1). According to some embodiments, pTR-CBA-FHL-1 comprises the nucleic acid sequence of SEQ ID NO: 7. According to some embodiments, pTR-CBA-FHL-1 consists of the nucleic acid sequence of SEQ ID NO: 7. According to some embodiments, the nucleic acid is at least 85% identical to SEQ ID NO: 7. According to some embodiments, the nucleic acid is at least 90% identical to SEQ ID NO: 7. According to some embodiments, the nucleic acid is at least 95%, 96%, 97%, or 98% identical to SEQ ID NO: 7. According to some embodiments, the nucleic acid is at least 99% identical to SEQ ID NO: 7.

SEQ ID NO: 7
ttggccactccctctctgcgcgctcgctcgctcactgaggccgggcgacca
aaggtcgcccgacgcccgggctttgcccgggcggcctcagtgagcgagcga
gcgcgcagagagggagtggccaactccatcactaggggttcctagatctga
attcggtaccctagttattaatagtaatcaattacggggtcattagttcat
agcccatatatggagttccgcgttacataacttacggtaaatggcccgcct
ggctgaccgcccaacgacccccgcccattgacgtcaataatgacgtatgtt
cccatagtaacgccaatagggactttccattgacgtcaatgggtggactat
ttacggtaaactgcccacttggcagtacatcaagtgtatcatatgccaagt
acgccccctattgacgtcaatgacggtaaatggcccgcctggcattatgcc
cagtacatgaccttatgggactttcctacttggcagtacatctacgtatta
gtcatcgctattaccatggtcgaggtgagccccacgttctgcttcactctc
cccatctcccccccctccccacccccaattttgtatttatttattttttaa
ttattttgtgcagcgatgggggcggggggggggggggggcgcgcgccaggc
ggggcggggcggggcgaggggcggggcggggcgaggcggagaggtgcggcg
gcagccaatcagagcggcgcgctccgaaagtttccttttatggcgaggcgg
cggcggcggcggccctataaaaagcgaagcgcgcggcgggcgggagtcgct
gcgacgctgccttcgccccgtgccccgctccgccgccgcctcgcgccgccc
gccccggctctgactgaccgcgttactcccacaggtgagcgggcgggacgg
cccttctcctccgggctgtaattagcgcttggtttaatgacggcttgtttc
ttttctgtggctgcgtgaaagccttgaggggctccgggagggccctttgtg
cgggggggagcggctcggggggtgcgtgcgtgtgtgtgtgcgtggggagcg
ccgcgtgcggcccgcgctgcccggcggctgtgagcgctgcgggcgcggcgc
ggggctttgtgcgctccgcagtgtgcgcgaggggagcgcggccgggggcgg
tgccccgcggtgcggggggggctgcgaggggaacaaaggctgcgtgcgggg
tgtgtgcgtgggggggtgagcagggggtgtgggcgcggcggtcgggctgta
acccccccctgcacccccctccccgagttgctgagcacggcccggcttcgg
gtgcggggctccgtacggggcgtggcgcggggctcgccgtgccgggcgggg
ggtggcggcaggtgggggtgccgggcggggcggggccgcctcgggccgggg
agggctcgggggaggggcgcggcggcccccggagcgccggcggctgtcgag
gcgcggcgagccgcagccattgccttttatggtaatcgtgcgagagggcgc
agggacttcctttgtcccaaatctgtgcggagccgaaatctgggaggcgcc
gccgcaccccctctagcgggcgcggggcgaagcggtgcggcgccggcagga
aggaaatgggcggggagggccttcgtgcgtcgccgcgccgccgtccccttc
tccctctccagcctcggggctgtccgcggggggacggctgccttcgggggg
gacggggcagggcggggttcggcttctggcgtgtgaccggcggctctagag
cctctgctaaccatgttcatgccttcttctttttcctacagctcctgggca
acgtgctggttattgtgctgtctcatcattttggcaaagaattcctcgaag
atctaggcaacgcgtctcgagtgatcagccaccatgagacttctagcaaag
attatttgccttatgttatgggctatttgtgtagcagaagattgcaatgaa
cttcctccaagaagaaatacagaaattctgacaggttcctggtctgaccaa
acatatccagaaggcacccaggctatctataaatgccgccctggatataga
tctcttggaaatattataatggtatgcaggaagggagaatgggttgctctt
aatccattaaggaaatgtcagaaaaggccctgtggacatcctggagatact
ccttttggtacttttacccttacaggaggaaatgtgtttgaatatggtgta
aaagctgtgtatacatgtaatgaggggtatcaattgctaggtgagattaat
taccgtgaatgtgacacagatggatggaccaatgatattcctatatgtgaa
gttgtgaagtgtttaccagtgacagcaccagagaatggaaaaattgtcagt
agtgcaatggaaccagatcgggaataccattttggacaagcagtacggttt
gtatgtaactcaggctacaagattgaaggagatgaagaaatgcattgttca
gacgatggtttttggagtaaagagaaaccaaagtgtgtggaaatttcatgc
aaatccccagatgttataaatggatctcctatatctcagaagattatttat
aaggagaatgaacgatttcaatataaatgtaacatgggttatgaatacagt
gaaagaggagatgctgtatgcactgaatctggatggcgtccgttgccttca
tgtgaagaaaaatcatgtgataatccttatattccaaatggtgactactca
cctttaaggattaaacacagaactggagatgaaatcacgtaccagtgtaga
aatggtttttatcctgcaacccggggaaatacagccaaatgcacaagtact
ggctggatacctgctccgagatgtaccttgaaaccttgtgattatccagac
attaaacatggaggtctatatcatgagaatatgcgtagaccatactttcca
gtagctgtaggaaaatattactcctattactgtgatgaacattttgagact
ccgtcaggaagttactgggatcacattcattgcacacaagatggatggtcg
ccagcagtaccatgcctcagaaaatgttattttccttatttggaaaatgga
tataatcaaaattatggaagaaagtttgtacagggtaaatctatagacgtt
gcctgccatcctggctacgctcttccaaaagcgcagaccacagttacatgt
atggagaatggctggtctcctactcccagatgcatccgtgtcagctttacc
ctctgacctgcagggcatgcgcggccgcgcggatccagacatgataagata
cattgatgagtttggacaaaccacaactagaatgcagtgaaaaaaatgctt
tatttgtgaaatttgtgatgctattgctttatttgtaaccattataagctg
caataaacaagttaacaacaacaattgcattcattttatgtttcaggttca
gggggaggtgtgggaggttttttagtcgactggggagagatctgaggaacc
cctagtgatggagttggccactccctctctgcgcgctcgctcgctcactga
ggccgcccgggcaaagcccgggcgtcgggcgacctttggtcgcccggcctc
agtgagcgagcgagcgcgcagagagggagtggccaac

“Minimal regulatory elements” are regulatory elements that are necessary for effective expression of a gene in a target cell. Such regulatory elements could include, for example, promoter or enhancer sequences, a polylinker sequence facilitating the insertion of a DNA fragment within a plasmid vector, and sequences responsible for intron splicing and polyadenlyation of mRNA transcripts. In a recent example of a gene therapy treatment for achromatopsia, the expression cassette included the minimal regulatory elements of a polyadenylation site, splicing signal sequences, and AAV inverted terminal repeats. See, e.g., Komaromy et al. The expression cassettes of the invention may also optionally include additional regulatory elements that are not necessary for effective incorporation of a gene into a target cell.

Vectors

The present invention also provides vectors that include any one of the expression cassettes discussed in the preceding section. In some embodiments, the vector is an oligonucleotide that comprises the sequences of the expression cassette. In specific embodiments, delivery of the oligonucleotide may be accomplished by in vivo electroporation (see, e.g., Chalberg, T W, et al. Investigative Ophthalmology & Visual Science, 46, 2140-2146 (2005) (hereinafter Chalberg et al., 2005)) or electron avalanche transfection (see, e.g., Chalberg, T W, et al. Investigative Ophthalmology & Visual Science, 47, 4083-4090 (2006) (hereinafter Chalberg et al., 2006)). In further embodiments, the vector is a DNA-compacting peptide (see, e.g., Farjo, R, et al. PLoS ONE, 1, e38 (2006) (hereinafter Farjo et al., 2006), where CK30, a peptide containing a cystein residue coupled to polyethylene glycol followed by 30 lysines, was used for gene transfer to photoreceptors), a peptide with cell penetrating properties (see Johnson, L N, et al., Cell-penetrating peptide for enhanced delivery of nucleic acids and drugs to ocular tissues including retina and cornea. Molecular Therapy, 16(1), 107-114 (2007) (hereinafter Johnson et al., 2007), Barnett, E M, et al. Investigative Ophthalmology & Visual Science, 47, 2589-2595 (2006) (hereinafter Barnett et al., 2006), Cashman, S M, et al. Molecular Therapy, 8, 130-142 (2003) (hereinafter Cashman et al., 2003), Schorderet, D F, et al. Clinical and Experimental Ophthalmology, 33, 628-635 (2005) (hereinafter Schorderet et al., 2005), Kretz, A, et al. Molecular Therapy, 7, 659-669 (2003) (hereinafter Kretz et al. 2003) for examples of peptide delivery to ocular cells), or a DNA-encapsulating lipoplex, polyplex, liposome, or immunoliposome (see e.g., Zhang, Y, et al. Molecular Vision, 9, 465-472 (2003) (hereinafter Zhang et al. 2003), Zhu, C, et al. Investigative Ophthalmology & Visual Science, 43, 3075-3080 (2002) (hereinafter Zhu et al. 2002), Zhu, C., et al. Journal of Gene Medicine, 6, 906-912. (2004) (hereinafter Zhu et al. 2004)).

In preferred embodiments, the vector is a viral vector, such as a vector derived from an adeno-associated virus, an adenovirus, a retrovirus, a lentivirus, a vaccinia/poxvirus, or a herpesvirus (e.g., herpes simplex virus (HSV)). See e.g., Howarth, J L et al., Using viral vectors as gene transfer tools. Cell Biol Toxicol 26:1-10 (2010). In the most preferred embodiments, the vector is an adeno-associated viral (AAV) vector.

Multiple serotypes of adeno-associated virus (AAV), including 12 human serotypes (AAV1, AAV2, AAV3, AAV4, AAV5, AAV6, AAV7, AAV8, AAV9, AAV10, AAV11, and AAV12) and more than 100 serotypes from nonhuman primates have now been identified. Howarth J L et al., 2010. In embodiments of the present invention wherein the vector is an AAV vector, the serotype of the inverted terminal repeats (ITRs) of the AAV vector may be selected from any known human or nonhuman AAV serotype. In preferred embodiments, the serotype of the AAV ITRs of the AAV vector is selected from the group consisting of AAV1, AAV2, AAV3, AAV4, AAV5, AAV6, AAV7, AAV8, AAV9, AAV10, AAV11, and AAV12. Moreover, in embodiments of the present invention wherein the vector is an AAV vector, the serotype of the capsid sequence of the AAV vector may be selected from any known human or animal AAV serotype. In some embodiments, the serotype of the capsid sequence of the AAV vector is selected from the group consisting of AAV1, AAV2, AAV3, AAV4, AAV5, AAV6, AAV7, AAV8, AAV9, AAV10, AAV11, and AAV12. In preferred embodiments, the serotype of the capsid sequence is AAV2. In some embodiments wherein the vector is an AAV vector, a pseudotyping approach is employed, wherein the genome of one ITR serotype is packaged into a different serotype capsid. See e.g., Zolutuhkin S. et al. Methods 28(2): 158-67 (2002). In preferred embodiments, the serotype of the AAV ITRs of the AAV vector and the serotype of the capsid sequence of the AAV vector are independently selected from the group consisting of AAV1, AAV2, AAV3, AAV4, AAV5, AAV6, AAV7, AAV8, AAV9, AAV10, AAV11, and AAV12.

In some embodiments of the present invention wherein the vector is a rAAV vector, a mutant capsid sequence is employed. Mutant capsid sequences, as well as other techniques such as rational mutagenesis, engineering of targeting peptides, generation of chimeric particles, library and directed evolution approaches, and immune evasion modifications, may be employed in the present invention to optimize AAV vectors, for purposes such as achieving immune evasion and enhanced therapeutic output. See e.g., Mitchell A. M. et al. AAV's anatomy: Roadmap for optimizing vectors for translational success. Curr Gene Ther. 10(5): 319-340.

AAV vectors can mediate long term gene expression in the retina and elicit minimal immune responses making these vectors an attractive choice for gene delivery to the eye.

### IV. Methods of Producing Viral Vectors

The present disclosure also provides methods of making a recombinant adeno-associated viral (rAAV) vectors comprising inserting into an adeno-associated viral vector any one of the nucleic acids described herein. According to some embodiments, the rAAV vector further comprises one or more AAV inverted terminal repeats (ITRs).

According to the methods of making an rAAV vector that are provided by the disclosure, the serotype of the capsid sequence and the serotype of the ITRs of said AAV vector are independently selected from the group consisting of AAV1, AAV2, AAV3, AAV4, AAV5, AAV6, AAV7, AAV8, AAV9, AAV10, AAV11, and AAV12. Thus, the disclosure encompasses vectors that use a pseudotyping approach, wherein the vector genome of one ITR serotype is packaged into a different serotype capsid. See e.g., Daya S. and Berns, K. I., Gene therapy using adeno-associated virus vectors. Clinical Microbiology Reviews, 21(4): 583-593 (2008) (hereinafter Daya et al.). Furthermore, in some embodiments, the capsid sequence is a mutant capsid sequence.

AAV Vectors

AAV vectors are derived from adeno-associated virus, which has its name because it was originally described as a contaminant of adenovirus preparations. AAV vectors offer numerous well-known advantages over other types of vectors: wildtype strains infect humans and nonhuman primates without evidence of disease or adverse effects; the AAV capsid displays very low immunogenicity combined with high chemical and physical stability which permits rigorous methods of virus purification and concentration; AAV vector transduction leads to sustained transgene expression in post-mitotic, nondividing cells and provides long-term gain of function; and the variety of AAV subtypes and variants offers the possibility to target selected tissues and cell types. Heilbronn R & Weger S, Viral Vectors for Gene Transfer: Current Status of Gene Therapeutics, in M. Schäfer-Korting (ed.), Drug Delivery, Handbook of Experimental Pharmacology, 197: 143-170 (2010) (hereinafter Heilbronn). A major limitation of AAV vectors is that the AAV offers only a limited transgene capacity (<4.9 kb) for a conventional vector containing single-stranded DNA.

AAV is a nonenveloped, small, single-stranded DNA-containing virus encapsidated by an icosahedral, 20 nm diameter capsid. The human serotype AAV2 was used in a majority of early studies of AAV. Heilbronn (2010). It contains a 4.7 kb linear, single-stranded DNA genome with two open reading frames rep and cap (“rep” for replication and “cap” for capsid). Rep codes for four overlapping nonstructural proteins: Rep78, Rep68, Rep52, and Rep40. Rep78 and Rep69 are required for most steps of the AAV life cycle, including the initiation of AAV DNA replication at the hairpin-structured inverted terminal repeats (ITRs), which is an essential step for AAV vector production. The cap gene codes for three capsid proteins, VP1, VP2, and VP3. Rep and cap are flanked by the 145 bp ITRs. The ITRs contain the origins of DNA replication and the packaging signals, and they serve to mediate chromosomal integration. The ITRs are generally the only AAV elements maintained in AAV vector construction.

To achieve replication, AAVs must be coinfected into the target cell with a helper virus. Grieger J C & Samulski R J, Adeno-associated virus as a gene therapy vector: Vector development, production, and clinical applications. Adv Biochem Engin/Biotechnol 99:119-145 (2005). Typically, helper viruses are either adenovirus (Ad) or herpes simplex virus (HSV). In the absence of a helper virus, AAV can establish a latent infection by integrating into a site on human chromosome 19. Ad or HSV infection of cells latently infected with AAV will rescue the integrated genome and begin a productive infection. The four Ad proteins required for helper function are E1A, E1B, E4, and E2A. In addition, synthesis of Ad virus-associated (VA) RNAs is required. Herpesviruses can also serve as helper viruses for productive AAV replication. Genes encoding the helicase-primase complex (UL5, UL8, and UL52) and the DNA-binding protein (UL29) have been found sufficient to mediate the HSV helper effect. In some embodiments of the present invention that employ rAAV vectors, the helper virus is an adenovirus. In other embodiments that employ rAAV vectors, the helper virus is HSV.

Making Recombinant AAV (rAA) Vectors

The production, purification, and characterization of the rAAV vectors of the present invention may be carried out using any of the many methods known in the art. For reviews of laboratory-scale production methods, see, e.g., Clark R K, Kidney Int. 61s:9-15 (2002); Choi V W et al., Current Protocols in Molecular Biology 16.25.1-16.25.24 (2007) (hereinafter Choi et al.); Grieger J C & Samulski R J, Adv Biochem Engin/Biotechnol 99:119-145 (2005) (hereinafter Grieger & Samulski); Heilbronn R & Weger S, in M. Schäfer-Korting (ed.), Drug Delivery, Handbook of Experimental Pharmacology, 197: 143-170 (2010) (hereinafter Heilbronn); Howarth J L et al., Cell Biol Toxicol 26:1-10 (2010) (hereinafter Howarth). The production methods described below are intended as non-limiting examples.

AAV vector production may be accomplished by cotransfection of packaging plasmids. Heilbronn. The cell line supplies the deleted AAV genes rep and cap and the required helpervirus functions. The adenovirus helper genes, VA-RNA, E2A and E4 are transfected together with the AAV rep and cap genes, either on two separate plasmids or on a single helper construct. A recombinant AAV vector plasmid wherein the AAV capsid genes are replaced with a transgene expression cassette (comprising the gene of interest, e.g., a CFH nucleic acid as described herein; a promoter; and minimal regulatory elements) bracketed by ITRs, is also transfected. These packaging plasmids can be transfected into adherent or suspension cell lines. According to some embodiments, these packaging plasmids are typically transfected into HEK 293 or HEK293T cells, a human cell line that constitutively expresses the remaining required Ad helper genes, E1A and E1B. This leads to amplification and packaging of the AAV vector carrying the gene of interest.

Multiple serotypes of AAV, including 12 human serotypes and more than 100 serotypes from nonhuman primates have now been identified. Howarth et al. The AAV vectors of the present invention may comprise capsid sequences derived from AAVs of any known serotype. As used herein, a “known serotype” encompasses capsid mutants that can be produced using methods known in the art. Such methods include, for example, genetic manipulation of the viral capsid sequence, domain swapping of exposed surfaces of the capsid regions of different serotypes, and generation of AAV chimeras using techniques such as marker rescue. See Bowles et al. Journal of Virology, 77(1): 423-432 (2003), as well as references cited therein. Moreover, the AAV vectors of the present invention may comprise ITRs derived from AAVs of any known serotype. Preferentially, the ITRs are derived from one of the human serotypes AAV1-AAV12. In some embodiments of the present invention, a pseudotyping approach is employed, wherein the genome of one ITR serotype is packaged into a different serotype capsid.

Preferentially, the capsid sequences employed in the present invention are derived from one of the human serotypes AAV1-AAV12. Recombinant AAV vectors containing an AAV5 serotype capsid sequence have been demonstrated to target retinal cells in vivo. See, for example, Komaromy et al. Therefore, in preferred embodiments of the present invention, the serotype of the capsid sequence of the AAV vector is AAV2. In other embodiments, the serotype of the capsid sequence of the AAV vector is AAV1, AAV2, AAV3, AAV4, AAV6, AAV7, AAV8, AAV9, AAV10, AAV11, or AAV12. Even when the serotype of the capsid sequence does not naturally target retinal cells, other methods of specific tissue targeting may be employed. See Howarth et al.

One possible protocol for the production, purification, and characterization of recombinant AAV (rAAV) vectors is provided in Choi et al. Generally, the following steps are involved: design a transgene expression cassette, design a capsid sequence for targeting a specific receptor, generate adenovirus-free rAAV vectors, purify and titer. These steps are summarized below and described in detail in Choi et al.

The transgene expression cassette may be a single-stranded AAV (ssAAV) vector or a “dimeric” or self-complementary AAV (scAAV) vector that is packaged as a pseudo-double-stranded transgene. Choi et al.; Heilbronn; Howarth. Using a traditional ssAAV vector generally results in a slow onset of gene expression (from days to weeks until a plateau of transgene expression is reached) due to the required conversion of single-stranded AAV DNA into double-stranded DNA. In contrast, scAAV vectors show an onset of gene expression within hours that plateaus within days after transduction of quiescent cells. Heilbronn. However, the packaging capacity of scAAV vectors is approximately half that of traditional ssAAV vectors. Choi et al. Alternatively, the transgene expression cassette may be split between two AAV vectors, which allows delivery of a longer construct. See e.g., Dyka et al. Hum Gene Ther. 2019 Sep. 30. A ssAAV vector can be constructed by digesting an appropriate plasmid (such as, for example, a plasmid containing the CFH gene) with restriction endonucleases to remove the rep and cap fragments, and gel purifying the plasmid backbone containing the AAVwt-ITRs. Choi et al. Subsequently, the desired transgene expression cassette can be inserted between the appropriate restriction sites to construct the single-stranded rAAV vector plasmid. A scAAV vector can be constructed as described in Choi et al.

Then, a large-scale plasmid preparation (at least 1 mg) of the rAAV vector and the suitable AAV helper plasmid and pXX6 Ad helper plasmid can be purified (Choi et al.). A suitable AAV helper plasmid may be selected from the pXR series, pXR1-pXR5, which respectively permit cross-packaging of AAV2 ITR genomes into capsids of AAV serotypes 1 to 12 and variants thereof. The appropriate capsid may be chosen based on the efficiency of the capsid's targeting of the cells of interest. For example, in a preferred embodiment of the present invention, the serotype of the capsid sequence of the rAAV vector is AAV2, because this type of capsid is known to effectively target retinal cells. Known methods of varying genome (i.e., transgene expression cassette) length and AAV capsids may be employed to improve expression and/or gene transfer to specific cell types (e.g., retinal cone cells). See, e.g., Yang G S, Journal of Virology, 76(15): 7651-7660.

Next, HEK293 or HEK293T cells are transfected with pXX6 helper plasmid, rAAV vector plasmid, and AAV helper plasmid. Choi et al. Subsequently the fractionated cell lysates are subjected to a multistep process of rAAV purification, followed by either CsCl gradient purification, or heparin sepharose column purification. The production and quantitation of rAAV virions may be determined using a dot-blot assay. In vitro transduction of rAAV in cell culture can be used to verify the infectivity of the virus and functionality of the expression cassette.

In addition to the methods described in Choi et al., various other transfection & purification methods for production of AAV may be used in the context of the present invention. For example, transient transfection methods are available, including methods that rely on a calcium phosphate precipitation or PEI protocol. The various purification methods include iodixanol gradient purification, affinity and/or ion-exchanger column chromatography.

In addition to the laboratory-scale methods for producing rAAV vectors, the present invention may utilize techniques known in the art for bioreactor-scale manufacturing of AAV vectors, including, for example, Heilbronn; Clement, N. et al. Human Gene Therapy, 20: 796-606. According to some embodiments, the method for producing rAAV vectors is carried out as described in Chulay et al. (Hum Gene Ther. 2011 February; 22(2):155-65), incorporated by reference in its entirety herewith.

### V. Methods of Treatment

The present disclosure provides methods of gene therapy for ocular disorders wherein rAAV particles, comprising AAV1-12, or portions or variants thereof, are delivered to the retina of a subject. According to one aspect, the disclosure provides methods of treating an ocular disease or disorder, comprising administering to a subject in need thereof an expression vector as described herein, wherein the expression vector comprises a nucleic acid encoding CFH, thereby treating the ocular disease or disorder in the subject. According to some embodiments, the expression vector further comprises two AAV terminal repeats. According to one aspect, the disclosure provides methods of preventing or stopping progression of an ocular disease or disorder, comprising administering to a subject in need thereof the expression vector as described herein, wherein the expression vector comprises a nucleic acid encoding CFH, thereby preventing or stopping progression of the ocular disease or disorder in the subject. According to another aspect, the disclosure provides methods of reversing the progression of an ocular disease or disorder, comprising administering to a subject in need thereof the expression vector as described herein, wherein the expression vector comprises a nucleic acid encoding CFH, thereby reversing the progression of the ocular disease or disorder in the subject. According to some embodiments, the expression vector further comprises at least two AAV terminal repeats. According to some embodiments, the ocular disease or disorder is associated with activation of the complement pathway. According to some embodiments, the ocular disease or disorder is retinal degeneration. According to some embodiments, the retinal degeneration is age related macular degeneration (AMD). According to some embodiments, the subject to be treated has manifested one or more signs or symptoms of an ocular disorder.

AMD is a complex, progressive eye disease which is the main reason for legal blindness and vision loss in the elderly worldwide (Pennington et al., Eye Vis. 2016, 3, 34). AMD results from both environmental and genetic factors, even though its actual etiology remains unclear. The number of individuals affected by AMD is about 196 million and projected to increase to 288 million in 2040 (Wong et al., Lancet Health 2014, 2, e106-e116). The main clinical symptom of AMD is the impairment of central vision, which may eventually result in complete vision loss. Advanced age is by definition the main AMD risk factor. Chronologically, AMD can be categorized as early and late. The early AMD is typified by the presence of, and increase in, deposits of extracellular debris between Bruch's membrane and RPE. These debris are called drusen, and their presence emerges with AMD progression (Joachim et al., Ophthalmology 2014, 121, 917-925). Late AMD may be manifested in two forms, atrophic (dry) and neovascular (wet). According to some embodiments, the AMD is dry AMD. According to some embodiments, the dry AMD is advanced dry AMD. The dry form of AMD is a more common form of AMD, accounting for 85 to 90 percent of all cases of age-related macular degeneration. It is characterized by a buildup of yellowish deposits called drusen beneath the retina and vision loss that worsens slowly over time. The condition typically affects vision in both eyes, although vision loss often occurs in one eye before the other. According to some embodiments, the AMD is wet AMD. The wet form of age-related macular degeneration is associated with severe vision loss that can worsen rapidly. This form of the condition is characterized by the growth of abnormal, fragile blood vessels underneath the macula. These vessels leak blood and fluid, which damages the macula and makes central vision appear blurry and distorted. Current wet AMD drug treatments focus on inhibiting vascular endothelial growth factor (VEGF), which stimulates blood vessel production. However, there remains a possibility of long-term effects of VEGF treatment. In mouse models, prolonged treatment with anti-VEGF therapy correlates with increased death of photoreceptors and their supporting cells within the retina (Ford et al., 2012. Invest. Ophthamol. Vis. Sci. 53, 7520-7527; Saint-Genie et al., 2008. PLoS ONE 3, e3554).

According to some embodiments, the disclosure further provides methods for treating an ocular disease or disorder (e.g. AMD) comprising administering any of the vectors of the invention to a subject in need of such treatment, thereby treating the subject.

In any of the methods of treatment, the vector can be any type of vector known in the art. In some embodiments, the vector is a non-viral vector, such as a naked DNA plasmid, an oligonucleotide (such as, e.g., an antisense oligonucleotide, a small molecule RNA (siRNA), a double stranded oligodeoxynucleotide, or a single stranded DNA oligonucleotide). In specific embodiments involving oligonucleotide vectors, delivery may be accomplished by in vivo electroporation (see e.g., Chalberg et al., 2005) or electron avalanche transfection (see e.g., Chalberg et al. 2006). In further embodiments, the vector is a dendrimer/DNA complex that may optionally be encapsulated in a water soluble polymer, a DNA-compacting peptide (see e.g., Farjo et al. 2006, where CK30, a peptide containing a cysteine residue coupled to poly ethylene glycol followed by 30 lysines, was used for gene transfer to photoreceptors), a peptide with cell penetrating properties (see Johnson et al. 2007; Barnett et al., 2006; Cashman et al., 2003; Schorder et al., 2005; Kretz et al. 2003 for examples of peptide delivery to ocular cells), or a DNA-encapsulating lipoplex, polyplex, liposome, or immunoliposome (see e.g., Zhang et al. 2003; Zhu et al. 2002; Zhu et al. 2004). According to some embodiments, the vector is a viral vector, such as a vector derived from an adeno-associated virus, an adenovirus, a retrovirus, a lentivirus, a vaccinia/poxvirus, or a herpesvirus (e.g., herpes simplex virus (HSV)). See e.g., Howarth. In preferred embodiments, the vector is an adeno-associated viral (AAV) vector.

According to some embodiments, the disclosure provides methods for treating an ocular disease or disorder (e.g. AMD) comprising administering a rAAV vector described herein, wherein the rAAV vector comprises a nucleic acid sequence encoding CFH.

According to some embodiments, the nucleic acid sequences described herein are directly introduced into a cell, where the nucleic acid sequences are expressed to produce the encoded product, prior to administration in vivo of the resulting recombinant cell. This can be accomplished by any of numerous methods known in the art, e.g., by such methods as electroporation, lipofection, calcium phosphate mediated transfection.

Pharmaceutical Compositions

According to some aspects, the disclosure provides pharmaceutical compositions comprising any of the vectors described herein, optionally in a pharmaceutically acceptable excipient.

As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolarity, encapsulating agents, pH buffering substances, and buffers. Such excipients include any pharmaceutical agent suitable for direct delivery to the eye which may be administered without undue toxicity. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N.J. 1991).

Generally, these compositions are formulated for administration by ocular injection. Accordingly, these compositions can be combined with pharmaceutically acceptable vehicles such as saline, Ringer's balanced salt solution (pH 7.4), and the like. Although not required, the compositions may optionally be supplied in unit dosage form suitable for administration of a precise amount.

Methods of Administration

According to the methods of treatment of the present invention, administering of a composition comprising a vector described herein can be accomplished by any means known in the art. According to some embodiments, the therapeutic compositions (e.g., nucleic acids encoding full length or truncated CFH proteins as described herein (e.g., tCFH1) are administered alone (i.e., without a vector for delivery). According to some embodiments, the administration is by ocular injection. According to some embodiments, the administration is by subretinal injection. Methods of subretinal delivery are known in the art. For example, see WO 2009/105690, incorporated herein by reference in its entirety. According to some embodiments, the compositions are directly injected into the subretinal space outside the central retina. In other embodiments, the administration is by intraocular injection, intravitreal injection, suprachoroidal, or intravenous injection. Administration of a vector to the retina may be unilateral or bilateral, and may be accomplished with or without the use of general anesthesia.

By safely and effectively transducing ocular cells (e.g., RPE) with a composition comprising a vector described herein, wherein the vector comprises a nucleic acid encoding CFH, the methods of the invention may be used to treat an individual; e.g., a human, having an ocular disorder (e.g., AMD), wherein the transduced cells produce CFH in an amount sufficient to treat the ocular disorder.

According to some embodiments, compositions may be administered by one or more subretinal injections, either during the same procedure or spaced apart by days, weeks, months, or years. According to some embodiments, multiple injections of a composition comprising a vector described herein, are no more than one hour, two hours, three hours, four hours, five hours, six hours, nine hours, twelve hours or 24 hours apart. According to some embodiments, multiple injections of a composition comprising a vector described herein, are about one month, two months, three months, four months, five months, six months, seven months, eight months, nine months, ten months, eleven months, twelve months or more apart. According to some embodiments, multiple injections of a composition comprising a vector described herein, are one year, two years, three years, four years, five years or more apart. According to some embodiments, multiple vectors may be used to treat the subject.

According to the methods of treatment of the present invention, the volume of vector delivered may be determined based on the characteristics of the subject receiving the treatment, such as the age of the subject and the volume of the area to which the vector is to be delivered. It is known that eye size and the volume of the subretinal or ocular space differ among individuals and may change with the age of the subject. According to some embodiments, the volume of the composition injected to the subretinal space of the retina is more than about any one of 1 μl, 2 μl, 3 μl, 4 μl, 5 μl, 6 μl, 7 μl, 8 μl, 9 μl, 10 μl, 15 μl, 20 μl, 25 μl, 50 μl, 75 μl, 100 μl, 200 μl, 300 μl, 400 μl, 500 μl, 600 μl, 700 μl, 800 μl, 900 μl, or 1 mL, or any amount therebetween. According to embodiments wherein the vector is administered subretinally, vector volumes may be chosen with the aim of covering all or a certain percentage of the subretinal or ocular space, or so that a particular number of vector genomes is delivered.

According to the methods of treatment of the present disclosure, the concentration of vector that is administered may differ depending on production method and may be chosen or optimized based on concentrations determined to be therapeutically effective for the particular route of administration. According to some embodiments, the concentration in vector genomes per milliliter (vg/ml) is selected from the group consisting of about 10 vg/ml, about 109 vg/ml, about 10¹⁰ vg/ml, about 10¹¹ vg/ml, about 10¹² vg/ml, about 10¹³ vg/ml, and about 10¹⁴ vg/ml or any amount therebetween. In preferred embodiments, the concentration is in the range of 10¹⁰ vg/ml-10¹³ vg/ml, delivered by subretinal injection or intravitreal injection in a volume of about 0.05 mL, about 0.1 mL, about 0.2 mL, about 0.4 mL, about 0.6 mL, about 0.8 mL, and about 1.0 mL.

According to some embodiments, one or more additional therapeutic agents may be administered to the subject. For example, anti-angiogenic agents (e.g., nucleic acids or polypeptides) may be administered to the subject.

The effectiveness of the compositions described herein can be monitored by several criteria. For example, after treatment in a subject using methods of the present disclosure, the subject may be assessed for e.g., an improvement and/or stabilization and/or delay in the progression of one or more signs or symptoms of the disease state by one or more clinical parameters including those described herein. Examples of such tests are known in the art, and include objective as well as subjective (e.g., subject reported) measures. For example, to measure the effectiveness of a treatment on a subject's visual function, one or more of the following may be evaluated: the subject's subjective quality of vision, the subject's dark adaptation, the subject's improved central vision function (e.g., an improvement in the subject's ability to read fluently and recognize faces), the subject's visual mobility (e.g., a decrease in time needed to navigate a maze), the subject's visual acuity (e.g., an improvement in the subject's Log MAR score), microperimetry (e.g., an improvement in the subject's dB score), dark-adapted perimetry (e.g., an improvement in the subject's dB score), fine matrix mapping (e.g., an improvement in the subject's dB score), Goldmann perimetry (e.g., a reduced size of scotomatous area (i.e., areas of blindness) and improvement of the ability to resolve smaller targets), flicker sensitivities (e.g., an improvement in Hertz), autofluorescence, and electrophysiology measurements (e.g., improvement in ERG). According to some embodiments, the visual function is measured by the subject's dark adaptation. The Dark Adaptation Test is a test used to determine the ability of the rod photoreceptors to increase their sensitivity in the dark. This test is a measurement of the rate at which the rod and cone system recover sensitivity in the dark following exposure to a bright light source. According to some embodiments, the visual function is measured by the subject's visual mobility. According to some embodiments, the visual function is measured by the subject's visual acuity. According to some embodiments, the visual function is measured by microperimetry. According to some embodiments, the visual function is measured by dark-adapted perimetry. According to some embodiments, the visual function is measured by ERG. According to some embodiments, the visual function is measured by the subject's subjective quality of vision.

In Vitro and In Vivo Models of AMD

Primary cultures of human fetal RPE (hfRPE) have been shown to be useful tools in AMD research because they model the function and metabolic activity of native RPE (Ablonczy et al., 2011. Invest. Ophthamol. Vis. Sci. 52, 8614-8620). Other RPE cell types used in AMD research include RPE derived from stem cells and the immortalized ARPE-19 cell line (Dunn et al., 1996, Exp. Eye Res. 62, 155-170).

The Cfh−/− mouse model is an in vivo model that can be used to study AMD. Complement factor H (CFH) plays an important regulatory role in the alternative pathway by preventing the binding of C3b with factor B and blocking the formation of C3 convertase (Pickering and Cook, 2008. Clin Exp Immunol. 2008 February; 151(2):210-30). Lack of CFH function leads to dysregulation of the alternative pathway resulting in low systemic levels of C3, deposition of C3 in glomerular basement membranes, and ultimately membranoproliferative glomerulonephritis (MPGN) Type II (Pickering and Cook, 2008). Mice genetically engineered to lack complement factor H also develop MPGN and retinal abnormalities reminiscent of AMD (Coffey et al., 2007. Proc Natl Acad Sci USA. 2007 Oct. 16; 104(42):16651-6; Pickering et al., 2002. Nat Genet. 2002 August; 31(4):424-8). At two years of age, these animals demonstrated decreased visual acuity as measured by water maze, reduction in rod-driven electroretinogram (ERG) a- and b-wave responses, increased subretinal autofluorescence, complement deposition in the retina, and disorganization of photoreceptor outer segments.

The transgenic CFH Y402H mouse model is an in vivo model that can be used to study AMD. To further elucidate the mechanisms by which CFH mutations contribute to AMD, transgenic mouse lines expressing the Y402H polymorphism under control of the human ApoE promoter were constructed (Ufret-Vincenty et al., 2010. Invest Ophthalmol Vis Sci. 2010 November; 51(11):5878-87). AMD-like symptom development in this mouse model also requires a high fat diet. The ApoE gene codes for apolipoprotein E, which is important in forming lipoproteins for lipid transport. At one year of age, these animals demonstrated a larger number of drusen-like deposits than seen in wild-type mice or Cfh−/− mice. Immunohistochemistry revealed increased numbers of microglial and macrophages in the subretinal space and electron microscopy showed thickening of Bruch's membrane and basement membrane deposition of C3d.

### VI. Kits

The rAAV compositions as described herein may be contained within a kit designed for use in one of the methods of the disclosure as described herein. According to some embodiments, a kit of the disclosure comprises (a) any one of the vectors of the disclosure, and (b) instructions for use thereof. According to some embodiments, a vector of the disclosure may be any type of vector known in the art, including a non-viral or viral vector, as described supra. According to some embodiments, the vector is a viral vector, such as a vector derived from an adeno-associated virus, an adenovirus, a retrovirus, a lentivirus, a vaccinia/poxvirus, or a herpesvirus (e.g., herpes simplex virus (HSV)). According to preferred embodiments, the vector is an adeno-associated viral (AAV) vector.

According to some embodiments, the kits may further comprise instructions for use. According to some embodiments, the kits further comprise a device for ocular delivery (e.g., intraocular injection, intravitreal injection, suprachoroidal, or intravenous injection) of compositions of rAAV vectors described herein. According to some embodiments, the instructions for use include instructions according to one of the methods described herein. The instructions provided with the kit may describe how the vector can be administered for therapeutic purposes, e.g., for treating an ocular disease or disorder (e.g., AMD). According to some embodiments wherein the kit is to be used for therapeutic purposes, the instructions include details regarding recommended dosages and routes of administration.

According to some embodiments, the kits further contain buffers and/or pharmaceutically acceptable excipients. Additional ingredients may also be used, for example preservatives, buffers, tonicity agents, antioxidants and stabilizers, nonionic wetting or clarifying agents, viscosity-increasing agents, and the like. The kits described herein can be packaged in single unit dosages or in multidosage forms. The contents of the kits are generally formulated as sterile and substantially isotonic solution.

All patents and publications mentioned herein are incorporated herein by reference to the extend allowed by law for the purpose of describing and disclosing the proteins, enzymes, vectors, host cells, and methodologies reported therein that might be used with the present disclosure. However, nothing herein is to be construed as an admission that the disclosure is not entitled to antedate such disclosure by virtue of prior disclosure.

The present disclosure is further illustrated by the following examples, which should not be construed as further limiting. The contents of all figures and all references, patents and published patent applications cited throughout this application, as well as the Figures, are expressly incorporated herein by reference in their entirety.

### EXAMPLES

### Example 1. Construct Design and Cloning

CFH is comprised of 20 CCPs which serve as binding sites for other proteins. It is known that the first 7 CCPs are important for complement regulation, as evidenced by the naturally occurring truncated CFH “FHL-1.” This truncated form of CFH (shown as SEQ ID NO: 7) is comprised of the first 7 CCPs and retains some function as a complement regulator. Thus, we aimed to truncate CFH by removing CCPs with no known function, or those with redundant binding sites. Two additional truncated constructs were generated for “Proof-of-Concept” in which CCPs known to be important for function were deleted to serve as a loss of function control.

The CBA promoter is a widely used, robust promoter that is capable of driving GOI expression across a multitude of cell types. The downside to the CBA promoter is its large size. In the present work, truncated versions of the CBA promoter were tested and used to save space in the AAV vector constructs.

The CBA promoter consists of a CMV ie enhancer, the core Chicken β-actin promoter, a short exon, and a long intron. The CMV ie enhancer and the intron are the largest segment of the full promoter and are not critical to promoter function, and instead act as enhancer elements. Thus, the aim was to truncate the promoter by deleting portions of the CMV ie enhancer and intron. In addition, a previously generated small CBA promoter (smCBA) was evaluated and included in the constructs.

pTR-CBA-flCFH: to generate the pTR-CBA-flCFH construct, the flCFH fragment was excised from pUC57-flCFH by NotI digestion and subsequently to generate pTR-CBA-flCFH
pTR-CBA-FHL-1: the same cloning strategy as that for pTR-CB-flCFH construction was used for pTR-CB-FHL-1 plasmid cloning. FHL-1 cDNA sequence (NCBI CCDS ID: 53452.1) plus appropriate cloning sites at both ends (NotI) was synthesized to create pTR-CBA_FHL-1
pTR-smCBA-flCFH: the pTR-smCBA-flCFH was constructed by replacing the the full CBA promoter in pTR-CBA-flCFH with the smCBA promoter. Both full length CBA and smCBA can be excised from their parental plasmids.
pTR-smCBA-tCFH1: the truncated CFH gene “tCFH1” is generated via PCR amplification and subsequently cloned into the pTR-smCBA backbone. Two PCR fragments (one contains CCPs 1 to 15 and the other contains CCPs 18 to 20) flanked by specific restriction sites for ligation were generated, digested with XhoI/KpnI or KpnI/NotI and then joined into pTR-smCBA backbone. SEQ ID NO: 2 shows the nucleic acid sequence of tCFH1.
pTR-smCBA-tCFH2 the truncated CFH gene “tCFH2” was generated via PCR amplification and subsequently cloned into the pTR-smCBA backbone. Two PCR fragments (one contains CCPs 1 to 4 and the other contains CCPs 18 to 20) were generated, digested with XhoI/KpnI or KpnI/NotI and then joined into pTR-smCBA backbone through 3-piece ligation. SEQ ID NO: 3 shows the nucleic acid sequence of tCFH2.
pTR-smCBA-tCFH3: the truncated CFH gene “tCFH3” was generated via PCR amplification and subsequently cloned into the pTR-smCBA backbone. Two PCR fragments (one contains CCPs 1 to 9 and the other contains CCPs 16 to 20) were generated, digested with XhoI/KpnI or KpnI/NotI and then joined into pTR-smCBA backbone through 3-piece ligation. SEQ ID NO: 4 shows the nucleic acid sequence of tCFH3.
pTR-smCBA-tCFH4: the truncated CFH gene “tCFH4” was generated via PCR amplification and subsequently cloned into the pTR-smCBA backbone. Two PCR fragments (one contains CCPs 1 to 7 and the other contains CCPs 18 to 20) were generated, digested with XhoI/KpnI or KpnI/NotI and then joined into pTR-smCBA backbone through 3-piece ligation. SEQ ID NO: 5 shows the nucleic acid sequence of tCFH4.

FIG. 1A is a schematic that shows the 20 complement control protein modules (CCPs) of full length human CFH (3696 bp). CCP modules are shown as ovals. Some CCPs have identified binding sites for other proteins as indicated. The construct pTR-CBA-flCFH comprises the full length human CFH. The high-risk polymorphism Y402H for AMD is located in CCP 7 which is also contained in the natural occurring variant FHL-1.

FIG. 1B is a schematic that shows CFH constructs that were engineered to have various CCP deleted. The construct pTR-smCBA-tCFH1 comprises the full length human CFH with CCP 16-17 deleted. The construct pTR-smCBA-tCFH2 comprises the full length human CFH with CCP 5-17 deleted. The construct pTR-smCBA-tCFH3 comprises the full length human CFH with CCP 10-15 deleted. The construct pTR-smCBA-tCFH4 comprises the full length human CFH with CCP 8-17 deleted. The construct pTR-CBA-FHL-1 comprises the natural occurring variant FHL-1. The two constructs, tCFH2 and tCFH4, were engineered to delete CCPs known to be important for complement cascade activity.

### Example 2. rAAV Production

Recombinant AAV vectors were produced by transfection of human embryonic kidney carcinoma 293 cells (HEK-293) as previously described (Xiao et al. (1998) J. Virol. 72:2224-2232). Transgenes were under the control of the chicken beta-actin (CBA) promoter or short version of CBA promoter (SmCBA). Virus was collected 68-76 hours post-transfection and purified twice using Iodixanol (IOD) gradient ultracentrifugation. After purification, virus was then concentrated and formulated in BSST (Alcon balanced salt solution with 0.014% Tween 20) using molecular weight cut off filters.

### Example 3. In Vitro Studies

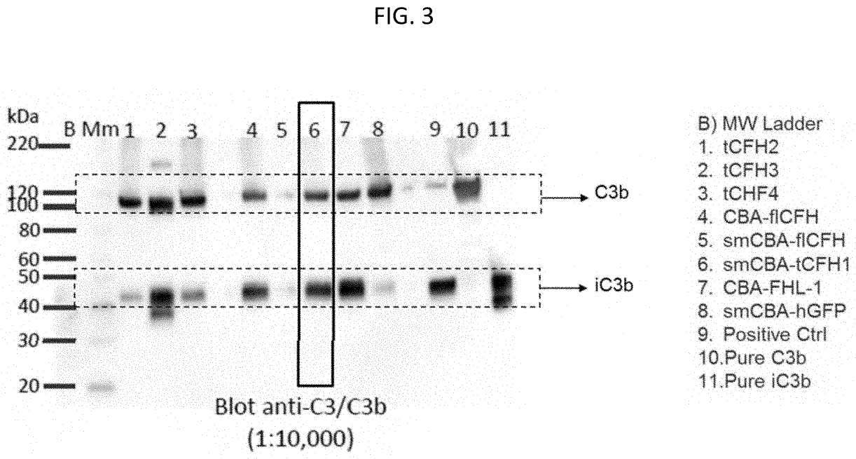

First, experiments were carried out with HEK293 cells transfected with the CFH variants. An ELISA assay was performed to determine CFH concentration (ng/ml) in the media. FIG. 2 is a graph that shows the expression of CFH variants following plasmid transfection of human embryonic kidney 293 (HEK293) cells. HEK293 cells were transfected with plasmids containing engineered CFH variants (pTR-CFH variants as shown in FIG. 1A). Cellular lysates were harvested 48 hours post transfection and stored at −80° C. until assayed. CFH concentration (ng/ml) was determined in the lysates.

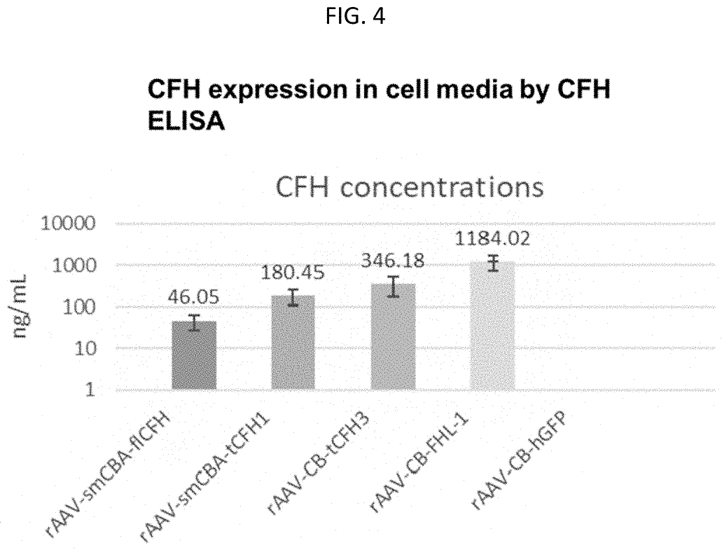

A cleavage assay was performed with the cell lysates to determine cleavage of human complement component C3b (C3b) by the CFH variants. FIG. 3 shows the results of Western blot with anti-C3/C3b antibody (Abcam, cat #129945) to assay cleavage of human C3b by the CFH variants. HEK293 cells were transfected with the plasmids and collected samples were stored as described in FIG. 2. FIG. 3 shows that efficient cleavage was observed in the smCBA-tCFH1 lane (lane 6, shown in box). Cleavage was absent or low by CFH variants smCBA-tCFH2 and smCBA-tCFH4. The same procedure may be carried out with cell supernatants, with similar expected results.

Based on these results, the following CFH variants were selected for AAV production: 1) pTR-smCBA-flCFH; 2) pTR-smCBA-tCFH1; 3) pTR-CBA-tCFH3; 4) pTR-CBA-FHL-1.

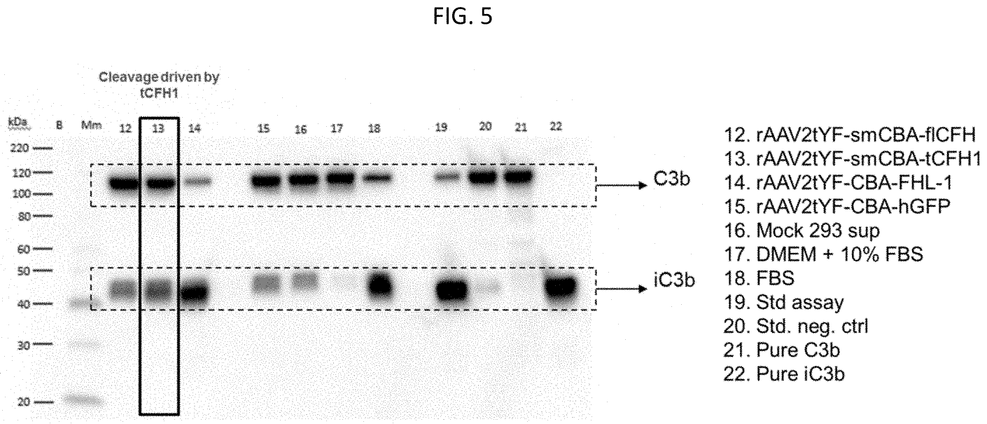

Next, rAAV-CFH infections of HEK293 cells were performed. An ELISA assay was used to measure CFH concentration (ng/ml) in the media. FIG. 4 is a graph that shows expression of CFH variants following rAAV infection of HEK293 cells with a multiplicity of infection (MOI) of 1×10⁴ vg. Samples were collected 72 hr post infection, and CFH concentration (ng/ml) was determined in the media. As shown in the graph, there was robust expression of the engineered CFH constructs 72 hours following rAAV-CFH infection of HEK293 cells.

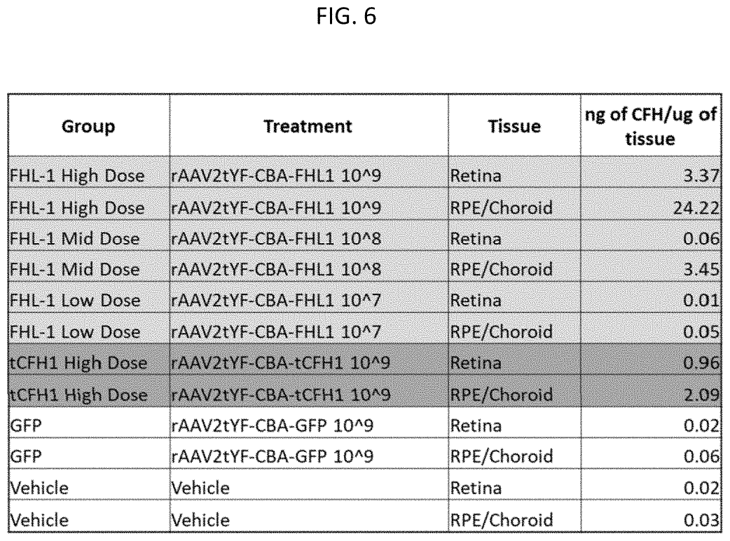

An assay was performed with the cell lysates to determine cleavage of human complement component C3b (C3b) by the rAAV expressed CFH variants. The results are shown in FIG. 5. As shown in FIG. 5, cleavage of C3b was most efficient by FHL-1, followed by tCFH1 and flCFH.

### Example 4. Hemolytic Experiments

The objective of this study was to evaluate the functionality of the rAAV-CFH variants in-vitro by evaluating the ability of each construct to induce lysis in rabbit erythrocytes/inhibit lysis of sheep erythrocytes.

Complement factor H (CFH) protein is composed of 20 complement control proteins (CCPs) each performing critical functions in alternate complement pathway activation. CCP 1-4 is important for C3b binding in the fluid phase (cleavage of C3b to iC3b) while CCP 19-20 bind glycosaminoglycans (GAGs) and sialic acids (SA) found on self-surfaces, in addition to binding C3b (Kerr et al., J. Biol. Chem. 2017; 292(32):13345-13360). Although all the CCPs work in a collaborative fashion to achieve complement activation on foreign surfaces and complement inhibition on self-surfaces, the absence of critical CCPs can deter chief functions of the CFH protein in a biological setting.

Because the CFH variants involve deletion of CCPs (as shown in FIG. 1A) from the wild-type CFH, testing them on the hemolysis assay helps to determine CCPs that are important for key functions such as fluid phase activity and membrane binding activity of CFH.

CFH plays a critical role in in-vitro activation of the alternate complement pathway in serum. Erythrocytes (RBCs) are sensitive to this complement activation causing them to lyse and release hemoglobin. Thus, lysis of RBCs turns the experimental diluent red, and the intensity of red color, which equates to the amount of hemoglobin released, can be measured photometrically at 415 nm.

The alternate complement regulatory proteins such as CFH are responsible for recognizing self from non-self. Foreign pathogens that do not express human regulatory proteins are recognized and destroyed by the alternate pathway (AP). Factor B, factor D and properdin proteins are unique to the alternate complement system. The AP pathway is capable of autoactivation via “tickover” of C3 that occurs spontaneously generating a conformational change in the protein. This modified C3 is capable of binding factor B leading to its conformation change. Modified factor B is cleaved by active serum protease factor D, generating Ba and Bb. The Bb protein remains associated with the complex, which can then cleave additional C3 molecules, generating C3b. C3b associates with factor B to generate more C3-convertase (C3bBb). The aforementioned steps are enhanced by serum protein Properdin, which is responsible for stabilizing protein: protein interactions. Thus, the AP can be initiated as an amplification loop when C3b binds factor B (Thurman et al., J Immunol 2006; 176(3):1305-1310). Thus, the absence of free factor B indicates the continuous activation of the complement pathway.

Since the AP pathway can be activated spontaneously, continuous control of the system is necessary. CFH is an active AP inhibitor and functions by binding C3b and converting it to inactive C3b or iC3b thereby preventing amplification of the AP loop. Thus, C3bBb convertase in not formed leaving free factor B in serum.

Addition of serum to RBCs causes activation of C3 and amplification of the AP loop, which proceeds without regulation when serum lacks a control protein such as CFH. Rabbit RBC membranes bind C3b efficiently and are shown to be resistant to inactivation of regulatory proteins since they lack sialic acid residues on the membrane (Fearon et al., J Exp Med 1977; 146(1): 22-33). Spontaneous fluid-phase activation of C3 occurs without regulation by CFH in CFH-depleted serum using up all free factor B. Due to the absence of free factor B, the AP loop is not amplified continuously and hence C3b is not formed. It has been shown that human serum depleted for CFH showed no C3 opsonization. Reconstitution of CFH-depleted serum to physiological levels resulted in C3 opsonization. (van der maten et al., JID 2016; 213:1820-1827). When CFH-depleted serum is added to rabbit RBCs along with CFH, AP activation leads to C3b deposition on the RBC membranes and AP progression leads to membrane attack complex (MAC) formation causing lysis of the RBCs. Induction of lysis by increasing concentrations of CFH is measured photometrically at 415 nm. 100% lysis is observed when CFH in serum is restored to physiological levels.

Normal human serum contains physiological levels of CFH. Hence, C3 activation proceeds under regulation of CFH. When normal human serum is added to antibody sensitized sheep erythrocytes, C3b binds and activates AP on the sheep RBC membrane. Sheep RBCs have sialic acid rich surface that can bind C-terminus of CFH (Yoshida et al., PLoS One 2015; 10(5): 1-21). Thus, when CFH is added to the reaction, it binds sheep RBC membranes efficiently blocking the C3 amplification loop. As a result, hemolysis of sheep RBCs is inhibited.

Sheep and rabbit RBCs help evaluate different functions of CFH. Hemolysis of sheep RBCs sheds light on the membrane binding activity of CFH modulated by CCPs 19-20. Hemolysis of rabbit RBCs sheds light on the fluid phase activity of CFH modulated majorly by CCPs 1-4. The CFH variants described herein were tested on both sheep and rabbit RBCs to evaluate their functionality.

Assay Conditions were as follows:

| Condition | Specification | Purpose |
|---|---|---|
| MgEGTA | 3 mM | Selective amplification |
| | Concentration | of AP pathway |
| | | on RBC surface |
| Red Blood cells | 2E6 cells/ | Test membrane binding |
| (Sheep/Rabbit) | reaction | activity of CFH |
| | | (Majorly conferred |
| | | by CCP 19-20) |
| Serum (Normal | 20% reaction | Supplies the necessary |
| human serum/ | volume | components |
| CFH-depleted | | for complement |
| serum) | | activation on RBCs |
| Factor H (CFH) | Purified CFH | Test protective/lysis |
| | protein, secreted | promoting activity of |
| | CFH from | CFH on RBCs |
| | transfection (3- | |
| | 5 ug/reaction) | |
| Optical Density | Absorbance | Absorbance is used |
| (OD) | read at | to measure levels of |
| | 415 nm | hemoglobin secreted by |
| | | the ruptured RBCs |
| | | due to AP activation |

Reactions were prepared my mixing RBCs with buffer containing MgEGTA, serum and CFH protein (purified, transfection or infection supernatants) and incubated at 37° C. for 30 min. MgEGTA is critical for selective and enhanced AP activation (des Prez at al., Infection and Immunity 1975; 11(6):1235-1243). The RBCs were centrifuged and optical density at 415 nm of the supernatant was measured for each of the reactions. Reactions were performed in duplicates.

HEK293T cells were transfected with the CFH plasmid variants and supernatants harvested 72 h post-transfection. CFH levels were measured in the supernatants prior to hemolysis assay.

HEK293T cells were infected with the rAAV-CFH variants and supernatants harvested 72 h post-infection. CFH levels were measured in the supernatants prior to hemolysis assay.

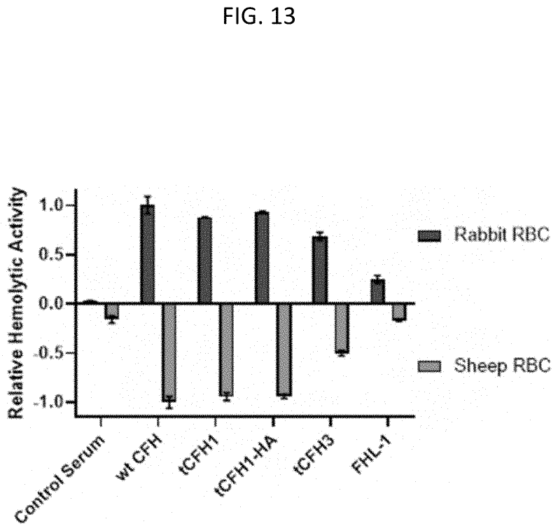

The results are shown in FIG. 13. As shown in FIG. 13, the rAAV-CFH variants had a lysis promoting function on Rabbit RBCs. The bars on top of the graph indicate the level of lysis induced by CFH on Rabbit RBCs. The levels of lysis are measured and plotted in the graph. Functionality of wild-type CFH is comparable to tCFH1 with and without the HA tag. This indicates that the truncated tCFH1 consists of all CCP regions critical for secretory function. The HA tag does not perturb tCFH1 functionality. FHL1 and tCFH3 functionality is relatively low when compared to cleavage assay, which measures the same secretory function of the CFH constructs. The cleavage assay uses <1 ng of C3b in the reaction and does not emulate the complexity of the AP pathway in serum. Thus, we observe a discrepancy in activity of FHL1 and tCFH3 in these two in-vitro assays. As also shown in FIG. 13, the rAAV-CFH variants had a protective function of CFH on Sheep RBCs. The bars on the bottom of the graph indicate the level of protective function exerted by CFH on Sheep RBCs. Control serum has inherent CFH levels that do not show a high degree of protection against lysis. However, the CFH constructs supplied in the reaction can bind sheep erythrocytes and block the AP pathway, thus inhibiting rupture of RBCs reflecting as reduced OD415 nm readings. Decreased lysis activity by FHL1 shows that CCPs 19-20 are critical for membrane binding activity. This makes FHL-1 a good proof of concept control. Functionality of wild-type CFH is comparable to tCFH1 with and without the HA tag. This indicates that the truncated tCFH1 consists of all CCP regions critical for secretory as well as membrane binding functionality. The HA tag does not perturb tCFH1 functionality. CCPs 10-15 have also been shown to play a role in C3b binding activity of CFH. This functionality also contributes to hemolytic activity of CFH. This explains the reduced functionality of tCFH3 in protecting sheep RBCs from lysis.

### Example 5. In Vivo Testing of FHL-1 and tCFH1 Constructs in Cfh−/− Mice

Activity of AAV-FHL-1 vector and AAV-tCFH1 was measured in CFH deficient (cfh−/−) mice. Mice were dosed subretinally with 1.0¹², 1.0¹¹ and 1.0¹⁰ vg/mL of AAV-FHL-1 or 1.0¹² vg/mL of AAV-tCFH1 into one eye. After 8 weeks, mice were terminated and the eyes were analyzed for CFH expression. Most eyes except for the eyes dosed with 1.0¹⁰ vg/mL were positive for CFH expression (a dose response was detected). The majority of eyes dosed with AAV-tCFH1 revealed FB fixation in addition to expression levels, while the eyes positive for FHL-1 did not.

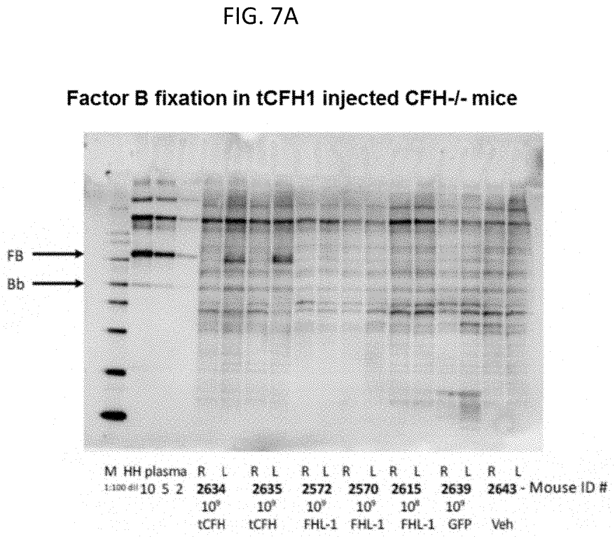

FIG. 6 is a table that shows the expression of tCFH1 or FHL-1 in cfh−/− mice after subretinal (SubR) injection. Both CFH variants FHL-1 and tCFH1 were expressed following subretinal dosing of rAAV vectors in cfh−/− mice. As shown in the results in the table, a dose response in FHL-1 expression was observed. Some animals were negative for expression of FHL-1 or tCFH1, which might have been due to unsuccessful injections. Expression level of tCFH1 or FHL-1 in RPE/choroid was found to be higher than the level in neural retina.

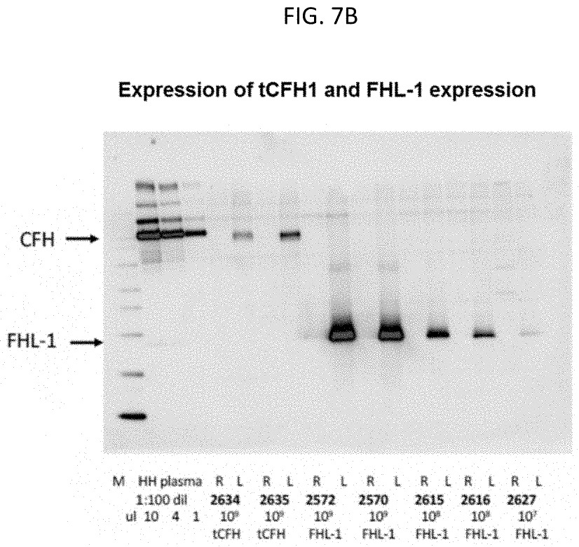

FIG. 7A and FIG. 7B show the results of Western blot to determine complement fixation (detection of Factor B (FB)) by tCFH1 variant. FIG. 7A shows factor B fixation in tCFH1 injected cjh−/− mice. FIG. 7B shows tCFH1 and FHL-1 expression. The results shown in FIG. 7A and FIG. 7B show that tCFH1 expression induced by rAAV-tCFH1 subretinal injection can fix factor B (FB) in RPE/choroid. The CFH variant FHL-1 did not show FB fixation. These results support the biological functionality of tCFH1 expressed by rAAV and is the first time that AAV expressed CFH variants show complement fixation.

### Example 6. tCFH1 Construct Dose Range Finding Study in cfh−/− Mice

Activity of AAV-tCFH1 at high, medium and low doses was measured in CFH deficient (cfh−/−) mice. The Table below shows the details of the study:

| | No of | Vector | Conc. | Dose | Vol. |
|---|---|---|---|---|---|
| Group | Animals | Left Eye | (vg/mL) | (vg/eye) | (μL) |
| 1 | 14 | rAAV2tYF-smCBA- | 1 × 10¹² | 1 × 10⁹ | 1 |
| (tCFH1 | | tCFH1 | | | |
| High Dose) | | | | | |
| 2 | 11 | rAAV2tYF-smCBA- | 5 × 10¹¹ | 5 × 10⁸ | 1 |
| (tCFH1 | | tCFH1 | | | |
| Mid Dose) | | | | | |
| 3 | 11 | rAAV2tYF-smCBA- | 1 × 10¹¹ | 1 × 10⁸ | 1 |
| (tCFH1 | | tCFH1 | | | |
| Low Dose) | | | | | |
| 4 | 2 | rAAV2tYF-CB-GFP | 1 × 10¹¹ | 1 × 10⁸ | 1 |
| (GFP High | | | | | |
| Dose) | | | | | |
| 5 | 2 | rAAV2tYF-CB-GFP | 1 × 10¹⁰ | 1 × 10⁷ | 1 |
| (GFP Low | | | | | |
| Dose) | | | | | |
| 6 | 2 | Vehicle (BSS + 0.014% | 0 | 0 | 1 |
| (Vehicle) | | Tween) | | | |

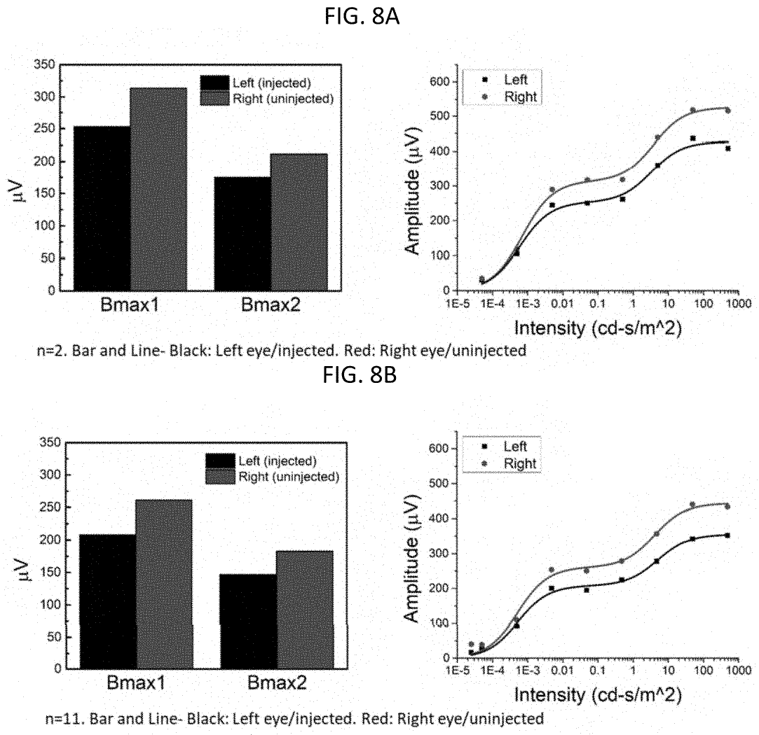

The electroretinogram (ERG) is a diagnostic test that measures the electrical activity of the retina in response to a light stimulus. The b wave of the ERG is widely believed to reflect the activation of on-bipolar cells. Prior to termination, scotopic b-wave ERG for vehicle and mid dose rAAV2tYF-smCBA-tCFHmice was measured. BMAX1 is the rod dominant component of scotopic ERG and BMAX2 is the cone dominant component of scotopic ERG. FIG. 8A shows the results in the mice dosed with the vehicle. FIG. 8B shows the results of the mice dosed with the tCFH1 Mid Dose. Some reduction of ERG in all injected eyes was seen, and was attributed to the surgical procedure. Minimal reduction in the mid and low dose groups shows vector safety. A more extensive reduction was seen in the high dose group, which indicates some vector toxicity at high dose.

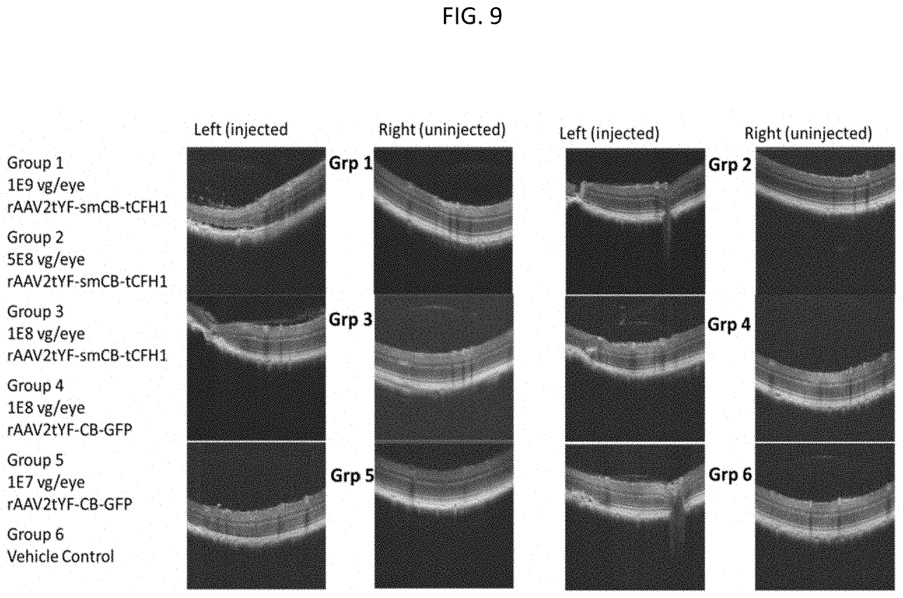

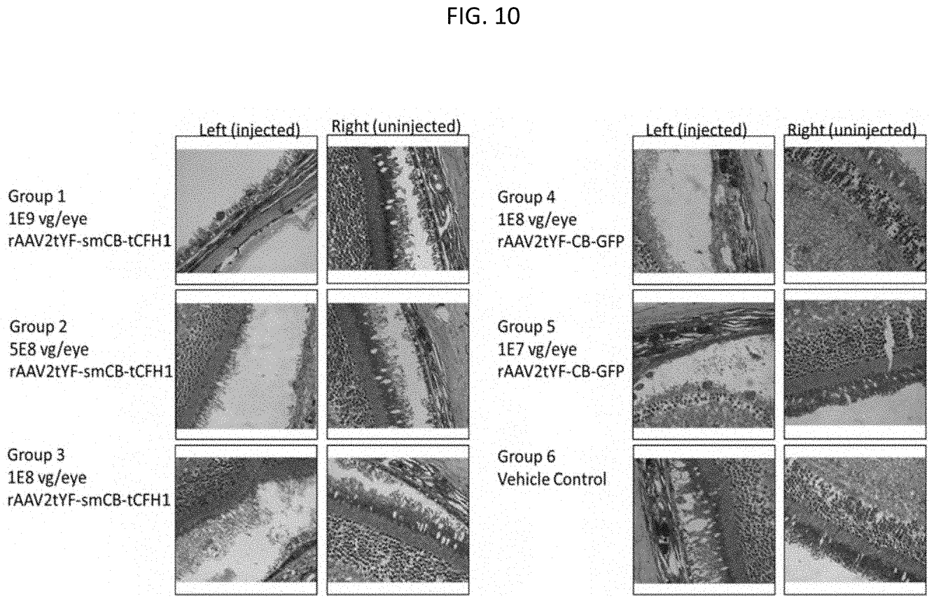

Optical coherence tomography (OCT) was used to generate in vivo, cross-sectional imagery of ocular tissues from left (injected) and right (uninjected) eyes prior to termination. The results for each of groups 1-6 are shown in FIG. 9. As shown in FIG. 9, some outer nuclear layer (ONL) thinning was observed in all injected eyes related to the surgical procedure. More extensive ONL thinning beyond the injected area was observed in the high dose group, which indicated some vector toxicity at the high dose. No changes in the ONL were observed in the uninjected eyes. Histological examination was performed on eyes after termination, and representative histological images from ocular tissues on left (injected) and right (uninjected) eyes for each of groups 1-6 are shown in FIG. 10. Some photoreceptor layer thinning and immune cell infiltrates in all injected eyes could be seen, and was related to surgical procedure. However, as shown in FIG. 10, more extensive photoreceptor layer thinning and immune cell infiltrates were seen in the high dose group, which indicated some vector toxicity at high dose. No changes were observed in the uninjected eyes.

Zonula occludens-1 (ZO-1) is a major structural protein of intercellular junctions. Next, ZO-1 staining was performed to assess retinal pigment epithelium (RPE) dyspmorphia, which would indicate RPE stress in each of groups 1-6. Uninjected eyes were used as a control. Flatmounts of RPE sheets obtained from each group and control were stained for ZO-1 and Hoechst (nuclei) and imaged with confocal microscopy. Some cell disorganization and immune cells were observed in all injected eyes, which were related to the surgical procedure (not shown). In the uninjected eyes, the RPE morphology resembled a regular hexagonal array of cells of uniform size throughout the retina. However, more extensive cell disorganization and immune cells were observed in high dose group, thus indicating some vector toxicity at high dose (not shown).

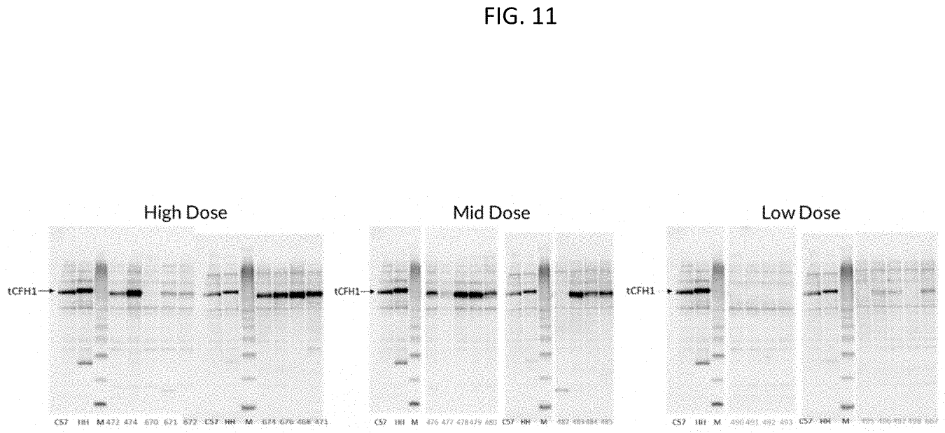

tCFH protein expression in cfh−/− mice injected with tCFH1 variant at low, mid and high doses was confirmed by Western blot. As shown in FIG. 11, a dose repose was seen in tCFH protein expression, with significant levels of tCFH1 expression observed with the high and mid dose. Minimal tCFH1 expression was observed with the low dose. Retinal extracts from normal C57B16 mice and transgenic mice expressing normal human CFH were used as positive controls. ELISA was also used to confirm tCFH1 expression. The results obtained from the ELISA experiments confirmed those from the Western Blot, where a dose repose was seen in tCFH protein expression, with significant levels of tCFH1 expression observed with the high and mid dose. Minimal tCFH1 expression was observed with the low dose. The Table below shows tCFH1 protein expression as determined by ELISA.

| | | | tCFH1 | |
|---|---|---|---|---|
| | | Animal | (ng/μg total protein | |
| Group | Dose | ID | RPE/Choroid/Sclera | Retina |
| High Dose | rAAVtYF- | 2674 | 0.296 | 0.137 |
| | smCBA-tCFH1 | 2676 | 0.532 | 0.148 |
| | (1 × 10 [symbol] vg/eye) | 2472 | 0.093 | 0.007 |
| | | 2474 | 0.178 | 0.094 |
| | | 2670 | 0.023 | 0.005 |
| | | 2671 | 0.060 | 0.061 |
| | | 2672 | 0.033 | 0.014 |
| | | 2468 | 0.389 | 0.418 |
| | | 2471 | 0.704 | 0.082 |
| Mid Dose | rAAVtYF- | 2476 | 0.038 | 0.027 |
| | smCBA-tCFH1 | 2477 | 0.027 | 0.037 |
| | (5 × 10 [symbol] vg/eye) | 2478 | 0.276 | 0.020 |
| | | 2479 | 0.282 | 0.012 |
| | | 2480 | 0.044 | 0.023 |
| | | 2482 | 0.012 | 0.003 |
| | | 2483 | 0.742 | 0.032 |
| | | 2484 | 0.217 | 0.116 |
| | | 2485 | 0.288 | 0.053 |
| Low Dose | rAAVtYF- | 2667 | 0.048 | 0.019 |
| | smCBA-tCFH1 | 2490 | 0.021 | 0.007 |
| | (1 × 10 [symbol] vg/eye) | 2491 | 0.070 | 0.005 |
| | | 2492 | 0.074 | 0.004 |
| | | 2495 | 0.040 | 0.005 |
| | | 2496 | 0.015 | 0.014 |
| | | 2497 | 0.020 | 0.012 |
| | | 2498 | 0.106 | 0.009 |

[symbol]

indicates data missing or illegible when filed

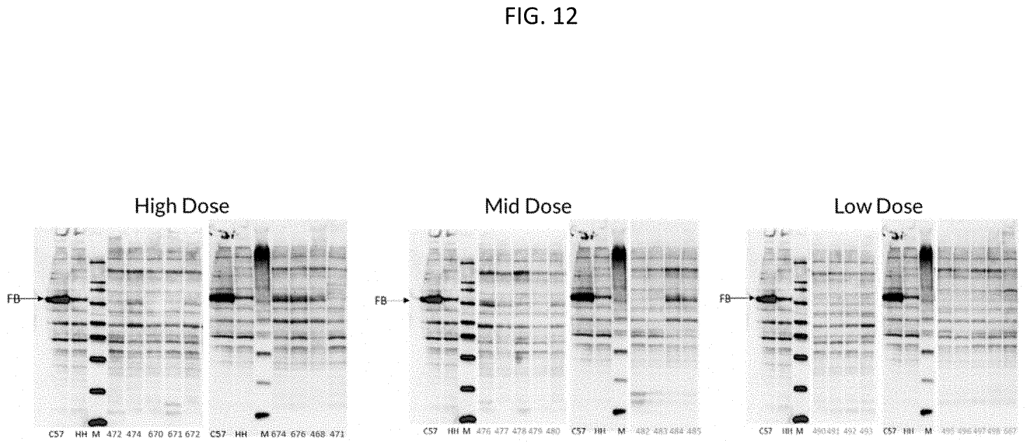

The complement system is active in the retina, RPE, and choroid under endogenous conditions. Factor B (FB) components have been detected in normal retinas, and genetic variations in several human complement components and regulators, such as factor B, have all been correlated with the occurrence of AMD (Gold B, et al. Nat Genet. 2006; 38:458-62). FIG. 12 shows the results of Western blot to determine Factor B (FB) complement fixation (detection of FB) inch cfh−/− mice injected with tCFH1 variant at various doses. As shown in FIG. 12, a dose response was observed, with better correlation between tCFH1 expression and FB fixation at higher doses. High dose and mid dose showed FB restoration, while no FB restoration was observed at low dose.

### Example 7. In Vivo Testing of rAAV-CFH Vectors in CFH H402 Mice

The objective of this study is to evaluate the efficacy of rAAV-CFH vectors in CFH H402 mice (CFH-HH:cfh−/−).

Complement factor H (CFH)single nucleotide polymorphisms (SNPs) have been reported as important genetic risk factors for age-related macular degeneration (AMD) pathogenesis. The Y402H polymorphism has been found to be the highest risk factor for AMD susceptibility. A transgenic mouse model that expresses full-length human CFH H402 in cfh−/− mice background (CFH-HH:cfh−/−) is used to test the effects of rAAV-CFH vectors. The mice are aged to 90 weeks and fed a high fat, cholesterol-enriched (HFC) diet. AMD-like phenotypes including vision loss, increased retinal pigmented epithelium (RPE) damage and increased sub-RPE deposit formation, are observed.

rAAV-hCFH vectors will be tested in this CFH-HH:cfh−/− murine model and evaluated for efficacy to rescue ERG, stop or reduce the RPE dysmorphogenesis and stop or reduce sub-RPE deposit accumulation.

### Study Design

rAAV-tCFH1 will be administered subretinally to the H402 mouse model (>90-week-old CFH-HH:cfh−/− mice on HFC diet) to evaluate their efficacy on inhibition of AMD-like pathological phenotypes including vision loss, retinal pigmented epithelium (RPE) damage and sub-RPE deposit formation. Only one eye will be injected.

A table of the study design is shown below.

| | | | | Study Read Outs | | | |
|---|---|---|---|---|---|---|---|
| | Treatment | | | | | | Western |
| | Left | | | | Flat | IHC and | And |
| Group | (1 μL) | Right | Dose | ERG | mount | Histology | ELISA |
| 1 | tCFH1 | None | High Dose | √ | √ | √ | √ |
| 2 | tCFH1 | None | Low Dose | √ | √ | √ | √ |
| 3 | FHL-1 | None | High Dose | √ | √ | √ | √ |
| 4 | Control | None | N/A | √ | √ | √ | √ |

*Immunohistochemisty

### EQUIVALENTS

Those skilled in the art will recognize or be able to ascertain using no more than routine experimentation, many equivalents to the specific embodiments of the invention described herein. Such equivalents are intended to be encompassed by the following claims.

1. A nucleic acid encoding a truncated complement factor H (CFH) protein, wherein the truncated CFH protein comprises 5 or more complement control protein modules (CCPs) selected from the group consisting of: CCP1, CCP2, CCP3, CCP4, CCP5, CCP6, CCP7, CCP8, CCP9, CCP10, CCP11, CCP12, CCP13, CCP14, CCP15, CCP16, CCP17, CCP18, CCP19 and CCP20.

2. (canceled)

3. The nucleic acid of claim 1, wherein the tCFH comprises SEQ ID NO: 2 or SEQ ID NO: 8.

4. A nucleic acid encoding a truncated CFH protein (tCFH), comprising a nucleotide sequence which is at least 85% identical to the nucleotide sequence selected from the group consisting of; SEQ ID NO: 2, SEO ID NO: 3, SEO ID NO: 4, SEO ID NO: 5 or SEQ ID NO: 8.

5. The nucleic acid of claim 1, wherein the nucleic acid encodes a truncated CFH protein (tCFH2) comprising at least CCP1, CCP2, CCP3, CCP4, CCP18, CCP19 and CCP20.

6. The nucleic acid of claim 5, comprising SEQ ID NO: 3.

7. (canceled)

8. The nucleic acid of claim 1, wherein the nucleic acid encodes a truncated CFH protein (tCFH3) comprising at least CCP1, CCP2, CCP3, CCP4, CCP5, CCP6, CCP7, CCP8, CCP9, CCP16, CCP17, CCP18, CCP19 and CCP20.

9. The nucleic acid of claim 8, comprising SEQ ID NO: 4.

10. (canceled)

11. The nucleic acid of claim 1, wherein the nucleic acid encodes a truncated CFH protein (tCFH4) comprising at least CCP1, CCP2, CCP3, CCP4, CCP5, CCP6, CCP7, CCP18, CCP19 and CCP20.

12. The nucleic acid of claim 11, comprising SEQ ID NO: 5.

13. (canceled)

14. A transgene expression cassette comprising
a promoter;
the nucleic acid of claim 1; and
minimal regulatory elements.

15. (canceled)

16. A nucleic acid vector comprising the expression cassette of claim 14.

17.-20. (canceled)

21. A mammalian cell comprising the vector of claim 16.

22. A method of making a recombinant adeno-associated viral (rAAV) vector comprising inserting into an adeno-associated viral vector a promoter and the nucleic acid of claim 1.

23. (canceled)

24. (canceled)

25. (canceled)

26. A method of treating an ocular disease or disorder, comprising administering to a subject in need thereof the vector of claim 16, thereby treating the ocular disease or disorder in the subject.

27. The method of claim 26, wherein the ocular disease or disorder is associated with activation of the complement pathway.

28. The method of claim 26, wherein the ocular disease or disorder is retinal degeneration.

29. The method of claim 28, wherein the retinal degeneration is age related macular degeneration (AMD).

30. The method of claim 29, wherein the AMD is wet AMD or dry AMD.

31. (canceled)

32. (canceled)

33. The method of claim 26, wherein the ocular disease or disorder is Geographic Atrophy (GA).

34. (canceled)

35. A method for delivering a heterologous nucleic acid to the eye of an individual comprising administering the vector of claim 16 to the retina of the individual.

36. (canceled)

37. (canceled)


SEQUENCE LISTING (10 sequences)

SEQ ID NO: 1 - DNA, 3696 bp, Homo sapiens
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatattataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattagatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 2 - DNA, 3351 bp, Artificial Sequence
  Description of Artificial Sequence Synthetic polynucleotide
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatattataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattagatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttggtacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 3 - DNA, 1359 bp, Artificial Sequence
  Description of Artificial Sequence Synthetic polynucleotide
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatattataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaaggtacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 4 - DNA, 2616 bp, Artificial Sequence
  Description of Artificial Sequence Synthetic polynucleotide
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatattataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattagatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagaggtaccccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 5 - DNA, 1722 bp, Artificial Sequence
  Description of Artificial Sequence Synthetic polynucleotide
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatattataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcggtaccacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 6 - DNA, 1350 bp, Artificial Sequence
  Description of Artificial Sequence Synthetic polynucleotide
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatattataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctga

SEQ ID NO: 7 - DNA, 3656 bp, Artificial Sequence
  Description of Artificial Sequence Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgagtgatcagccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatattataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctgacctgcagggcatgcgcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggcca

SEQ ID NO: 8 - DNA, 3372 bp, Artificial Sequence
  Description of Artificial Sequence Synthetic polynucleotide
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatcgccctggatatagatctcttggaaatatcataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattacggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggcacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 9 - protein, 1116 aa, Artificial Sequence
  Description of Artificial Sequence Synthetic polypeptide
MRLLAKIICLMLWAICVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSIRCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLGTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 10 - protein, 1114 aa, Artificial Sequence
  Description of Artificial Sequence Synthetic polypeptide
MRLLAKIICLMLWAICVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR